(19) United States
(12) Patent Application Publication
(10) Pub. No.: US 2006/0188515 A1
(43) Pub. Date: Aug. 24, 2006

(54) Polypeptides for inducing a protective immune response against staphylococcus aureus

(76) Inventors: Annaliesa S. Anderson, Doylestown, PA (US); Nelly Kuklin, Collegeville, PA (US); Kathrin Uwe Jansen, South San Francisco, CA (US)

Correspondence Address: MERCK AND CO., INC, P O BOX 2000, RAHWAY, NJ, 07065-0907, US

(21) Appl. No.: 10/564,375

(22) PCT Filed: Jul. 22, 2004
(86) PCT No.: PCT/US04/23522

§ 371 Date: Jan. 12, 2006

Related U.S. Application Data
(60) Provisional application No. 60/489,840, filed on Jul. 24, 2003.

(51) Int. Cl. A61K 39/02; C07K 14/31

(52) U.S. Cl. 424/190.1; 530/350

(57) ABSTRACT

The present invention features hybrid polypeptides providing ORF0657n and ORFO190 epitopes, nucleic acid encoding for the different polypeptides, and a method for evaluating the ability of an immunogen to produce a protective immune response against Staphylococcus infection. ORF0657n and ORF0190 are both S. aureus proteins.

### CROSS-REFERENCE TO RELATED APPLICATIONS

The present application claims priority to provisional application U.S. Ser. No. 60/489,840, filed Jul. 24, 2003, hereby incorporated by reference herein.

### BACKGROUND OF THE INVENTION

The references cited throughout the present application are not admitted to be prior art to the claimed invention.

Staphylococcus aureus is a pathogen responsible for a wide range of diseases and conditions. Examples of diseases and conditions caused by S. aureus include bacteremia, infective endocarditis, folliculitis, furuncle, carbuncle, impetigo, bullous impetigo, cellulitis, botryomyosis, toxic shock syndrome, scalded skin syndrome, central nervous system infections, infective and inflammatory eye disease, osteomyletitis and other infections of joints and bones, and respiratory tract infections. (The Staphylococci in Human Disease, Crossley and Archer (eds.), Churchill Livingstone Inc. 1997.)

Immunological based strategies can be attempted to control S. aureus infections and the spread of S. aureus. Immunological based strategies include passive and active immunization. Passive immunization employs immunoglobulins targeting S. aureus. Active immunization induces immune responses against S. aureus.

Potential S. aureus vaccines target S. aureus polysaccharides and polypeptides. Targeting can be achieved using S. aureus polysaccharides or polypeptides as possible vaccine components. Examples of polysaccharides that may be employed as possible vaccine components include S. aureus type 5 and type 8 capsular polysaccharides. (Shinefield et al., N. Eng. J. Med. 346:491-496, 2002.) Examples of polypeptides that may be employed as possible vaccine components include collagen adhesin, fibrinogen binding proteins, and clumping factor. (Mamo et al., FEMS Immunology and Medical Microbiology 10:47-54, 1994, Nilsson et al., J. Clin. Invest. 101:2640-2649, 1998, Josefsson et al., The Journal of Infectious Diseases 184:1572-1580, 2001.)

Information concerning S. aureus polypeptide sequences has been obtained from sequencing the S. aureus genome. (Kuroda et al., Lancet 357:1225-1240, 2001, Baba et al., Lancet 359:1819-1827, 2000, Kunsch et al., European Patent Publication EP 0 786 519, published Jul. 30, 1997.) To some extent bioinformatics has been employed in efforts to characterize polypeptide sequences obtained from genome sequencing. (Kunsch et al., European Patent Publication EP 0 786 519, published Jul. 30, 1997.)

Techniques such as those involving phage display technology and sera from infected patients can be used in an effort to identify genes coding for potential antigens. (Foster et al., International Publication Number WO 01/98499, published Dec. 27, 2001.)

### SUMMARY OF THE INVENTION

The present invention features hybrid polypeptides providing ORF0657n and ORFO190 epitopes, nucleic acid encoding for the different polypeptides, and a method for evaluating the ability of an immunogen to produce a protective immune response against Staphylococcus infection. ORF0657n and ORF0190 are both S. aureus proteins.

A preferred use of hybrid and ORF0657n polypeptides is to induce a protective immune response against S. aureus. Protective immunity or immune response provides a detectable level of protection against S. aureus infection. The level of protection can be assessed using animal models such as those described in Example 1 infra.

Thus, a first aspect of the present invention describes a hybrid polypeptide immunogen comprising a modified ORF0657n sequence segment at least about 100 amino acids in length. An ORF0657n “sequence segment” provides all or a portion of an ORF0657n protein as a references sequence.

Hybrid polypeptides comprise a modified ORF0657n sequence segment containing one or more alterations increasing sequence similarity to SEQ ID NO: 1 (ORF0190). The modified ORF0657n sequence segment comprises one or more alterations increasing sequence similarity to SEQ ID NO: 1. An amino acid alteration is an addition, deletion, or substitution. Different combinations of amino acids alterations may be present.

Reference to an alteration or modification is a structural distinction between a reference sequence and is not a method of production limitation. Altered and modified sequences can be produced, for example, by altering a preexisting sequence or synthesizing a desired sequence.

Reference to “polypeptide” includes salt forms and does not provide a size limitation or function. A polypeptide may include, for example, a protein or a fragment thereof.

Reference to “immunogen” indicates the ability to produce an immune response. An immunogen contains one or more polypeptide regions, and may also contain one or more regions that are not polypeptides and/or one or more moieties that are not amino acids.

Another aspect of the present invention features a method of making a hybrid polypeptide comprising a modified ORF0657n sequence. The method comprises the step of introducing one or more alterations into a ORF0657n sequence segment at least about 100 amino acids in length, wherein at least one of the alterations increases sequence similarity to SEQ ID NO: 1.

Another aspect of the present invention describes an immunogen comprising an amino acid sequence that provides protective immunity against S. aureus. The immunogen comprises an amino acid sequence providing a hybrid polypeptide and one or more additional regions or moieties covalently joined at the carboxyl terminus or amino terminus, wherein each region or moiety is independently selected from a region or moiety having at least one of the following properties: enhances the immune response, facilitates purification, or facilitates polypeptide stability.

Reference to “additional region or moiety” indicates a region or moiety different from a ORF0657n or ORF0190 related polypeptide which would be produced in a biological host, such as a prokaryotic or eukaryotic host. The additional region or moiety can be, for example, an additional polypeptide region or a non-peptide region.

Another aspect of the present invention describes a composition able to induce a protective immune response in a patient. The composition comprises an immunologically effective amount of an immunogen that induces protective immunity against S. aureus and a pharmaceutically acceptable carrier.

An immunologically effective amount is an amount sufficient to provide protective immunity against S. aureus infection. The amount should be sufficient to significantly prevent the likelihood or severity of a S. aureus infection.

Another aspect of the present invention describes a method of inducing a protective immune response in a patient against S. aureus. The method comprises the step of administering to the patient an immunologically effective amount of an immunogen.

Another aspect of the present invention describes a nucleic acid comprising a nucleotide sequence encoding a polypeptide described herein. In a preferred embodiment the nucleic acid is a recombinant nucleic acid. Recombinant nucleic acid is nucleic acid that by virtue of its sequence or form does not occur in nature.

Another aspect of the present invention describes a cell comprising a recombinant gene encoding a polypeptide described herein. A recombinant gene contains recombinant nucleic acid encoding a polypeptide along with regulatory elements for proper transcription and processing.

Another aspect of the present invention describes a method for evaluating the efficacy of an immunogen to produce a protective immune response against Staphylococcus. The method comprises the steps of:

(a) immunizing an animal model with an immunogen;

(b) challenging the immunized animal model with a Staphylococcus challenge at a potency that provides about 80 to 90% death in an non-immunized animal model over a period of about 7-10 days starting on the first or second day, wherein the Staphylococcus challenge is produced from Staphylococcus grown to stationary phase, and the Staphylococcus challenge is intravenously introduced into the animal; and

(c) measuring the ability of the immunogen to provide protective immunity.

Unless particular terms are mutually exclusive, reference to “or” indicates either or both possibilities. Occasionally phrases such as “and/or” are used to highlight either or both possibilities.

Reference to open-ended terms such as “comprises” allows for additional elements or steps. Occasionally phrases such as “one or more” are used with or without open-ended terms to highlight the possibility of additional elements or steps.

Unless explicitly stated reference to terms such as “a” or “an” is not limited to one. For example, “a cell” does not exclude “cells”. Occasionally phrases such as one or more are used to highlight the presence of a plurality.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIGS. 1A, 1B and 1C illustrate a sequence comparison between ORF0190 (SEQ ID NO: 1), 0657n (SEQ ID NO: 2), 0657nHybrid1 (SEQ ID NO: 8), 0657nHybrid2 (SEQ ID NO: 9), and 0657nHybrid3 (SEQ ID NO: 10). SEQ ID NOs: 8, 9 and 10 are examples of hybrid polypeptides comprising a modified ORF0657n sequence segment.

FIGS. 2A-2L provide examples of different hybrid sequences (SEQ ID NOs: 8-43).

FIG. 3 illustrates forward and reverse PCR primer sequences for amplifying a nucleic acid sequence to encode a mutated form of ORF0657n. ORF0657n amino acids are indicated in non italics; added amino acids (the initiator M and G) coded by the forward primer are indicated by italics. Restriction sites are underlined. Non-expressed regions are in parenthesis. SEQ ID NOs: 46 and 48 are forward and reverse primer sequences. SEQ ID NOs: 47 and 49 are the amino acid sequences encoded by the forward and reverse primers.

FIGS. 4A and 4B illustrate translation of a cloned and expressed mutated form of ORF0657n. The mutated form of ORF0657n contains modifications to facilitate cloning and purification. FIG. 4A illustrates the expressed sequence including additional histidine residues (SEQ ID NO: 44). FIG. 4B illustrates an abbreviated alignment of the native and mutated forms of ORF0657n showing differences between the two forms.

FIG. 5 illustrates survival data using a mutated ORF0657n in aluminum hydroxyphosphate adjuvant (AHP).

### DETAILED DESCRIPTION OF THE INVENTION

The present invention features a hybrid polypeptide immunogen comprising a modified ORF0657n sequence segment, nucleic acid encoding such polypeptides and a method for evaluating the ability of an immunogen to produce a protective immune response against Staphylococcus infection. Hybrid polypeptides polypeptides have therapeutic and diagnostic applications, such as being used to provide protective immunity against a S. aureus infection, being using to generate antibodies to detect the presence of S. aureus, and being used to generate therapeutic antibodies targeting S. aureus.

Hybrid Polypeptides

Hybrid polypeptides comprise a modified ORF0657n sequence segment containing one or more alterations increasing sequence similarity to ORF0190 (SEQ ID NO: 1). The hybrid polypeptide contains one or more epitopes for ORF0657n and ORF0190.

Hybrid polypeptides can be designed taking into account the similarity and differences between ORF0657n and ORF0190 proteins sequences. An amino acid alignment of ORF0657n (SEQ ID NO: 2) and ORF0190 (SEQ ID NO: 1) revealed a central region having a high degree of homology between the two proteins. The region for ORF0657n spans 327 amino acids from position 122 to 448. The ORF0190 homologous region spans 328 amino acids from position 323 to 650. Within the central region, 64% of the amino acids were identical and there was an over all level of 80% similarity.

Sequence similarity was determined using a local alignment tool utilizing the program lalign (developed by Huang and Miller, Adv. Appl. Math. 12:337-357, 1991, for the <<sim>> program). The options and environment variables are:-f # Penalty for the first residue a gap (-14 by default); -g # Penalty for each additional residue in a gap (-4 by default)-s str (SMATRIX) the filename of an alternative scoring matrix file. For protein sequences, PAM250 is used by default-w # (LINEN) output line length for sequence alignments (60).

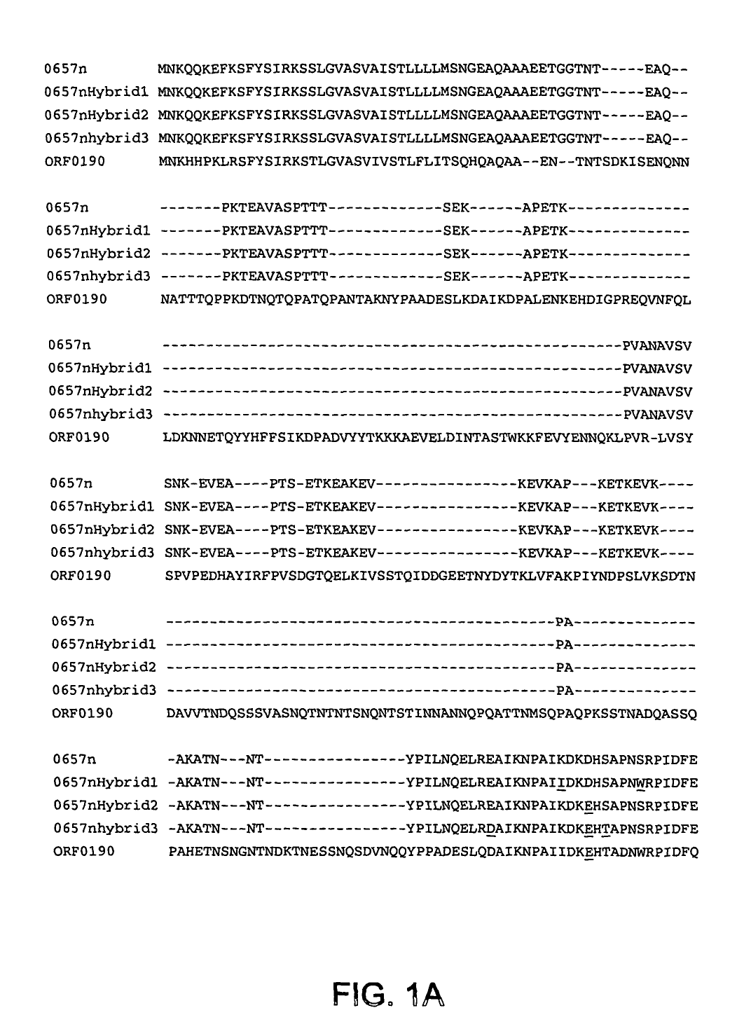

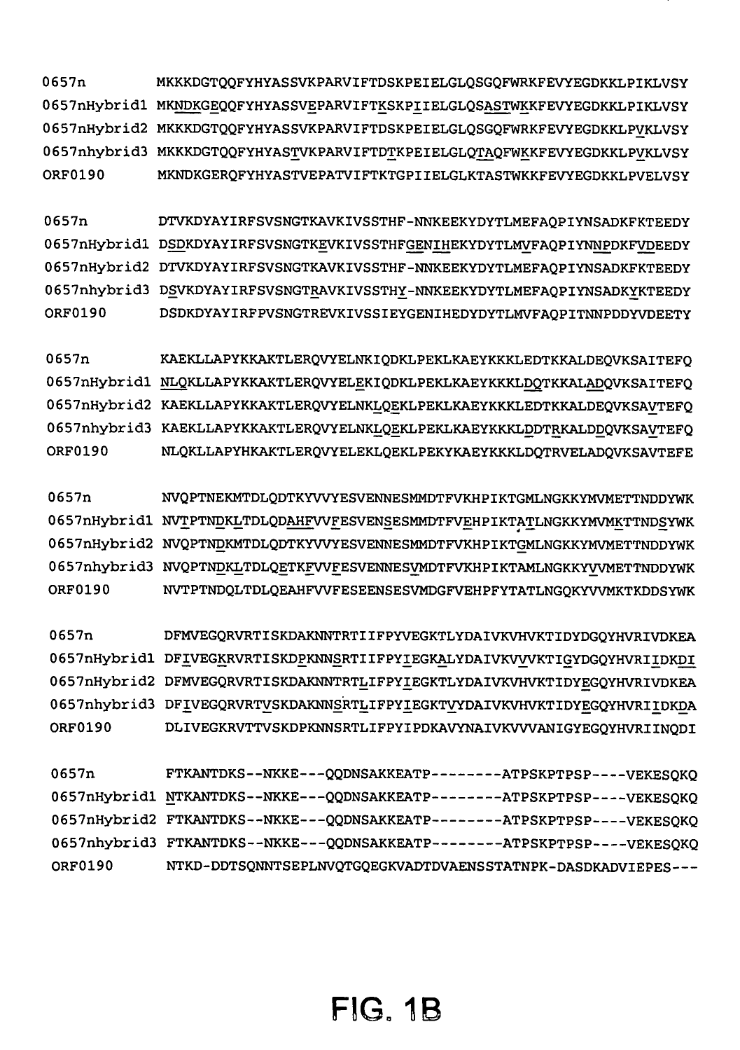

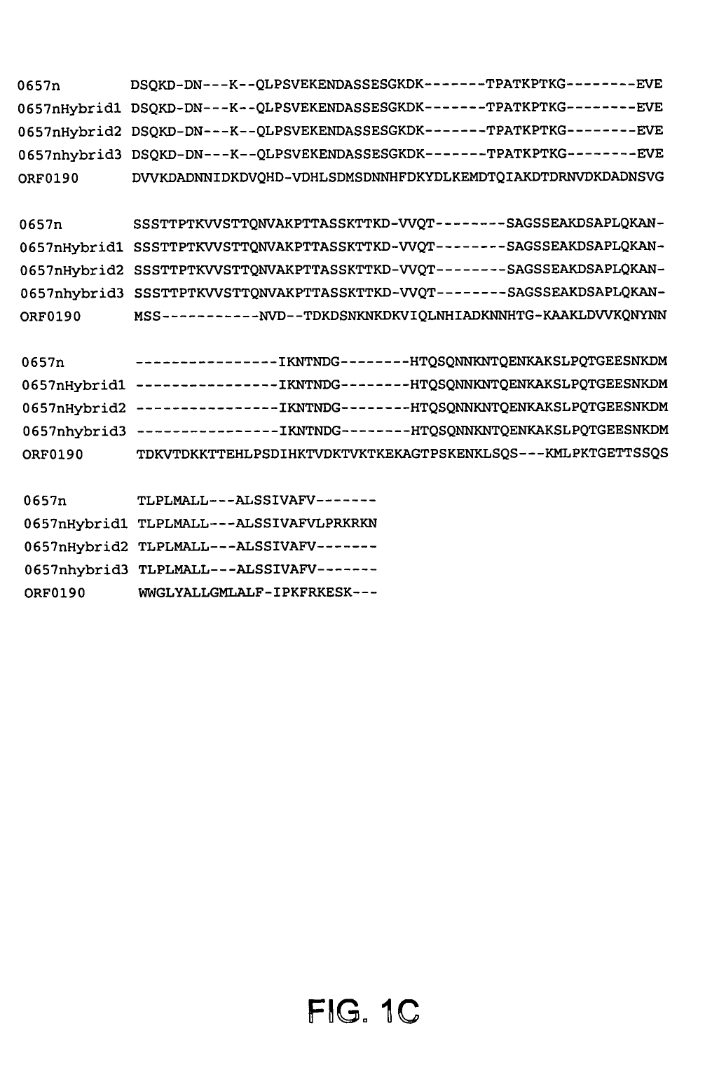

FIGS. 1A-1C illustrate the central region of homology between an ORF0657n (SEQ ID NO: 2) and ORF0190 (SEQ ID NO: 1) and provides examples of hybrid polypeptides that can be designed taking into account ORF0657n and ORF0190 sequences. Additional hybrid polypeptides can be obtained based on the ORF0657n and ORF0190 sequence alignment provided in FIGS. 1A-1C, and alignments produced using other ORF0657 sequences.

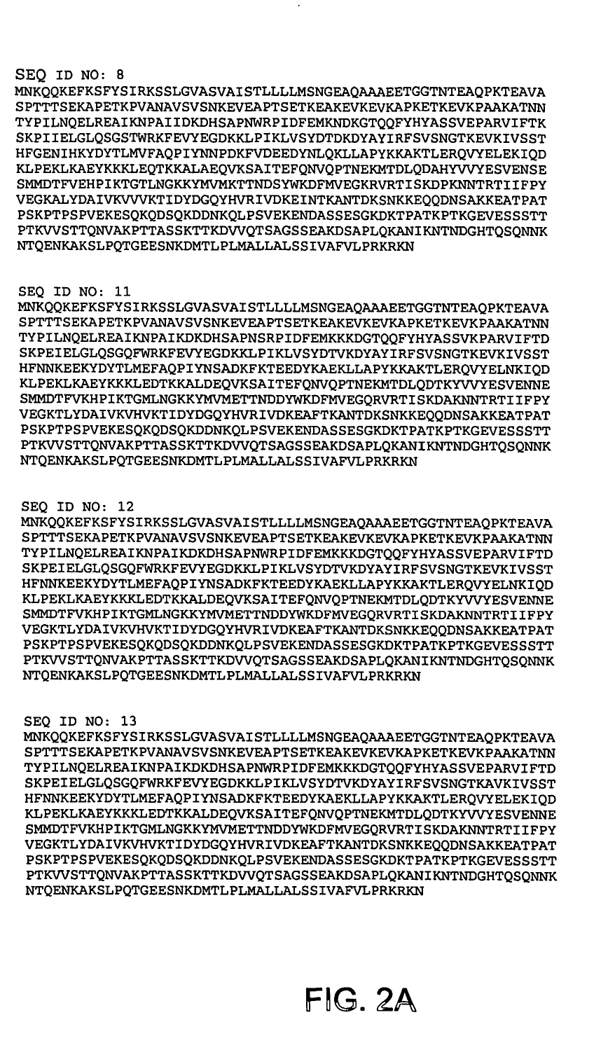

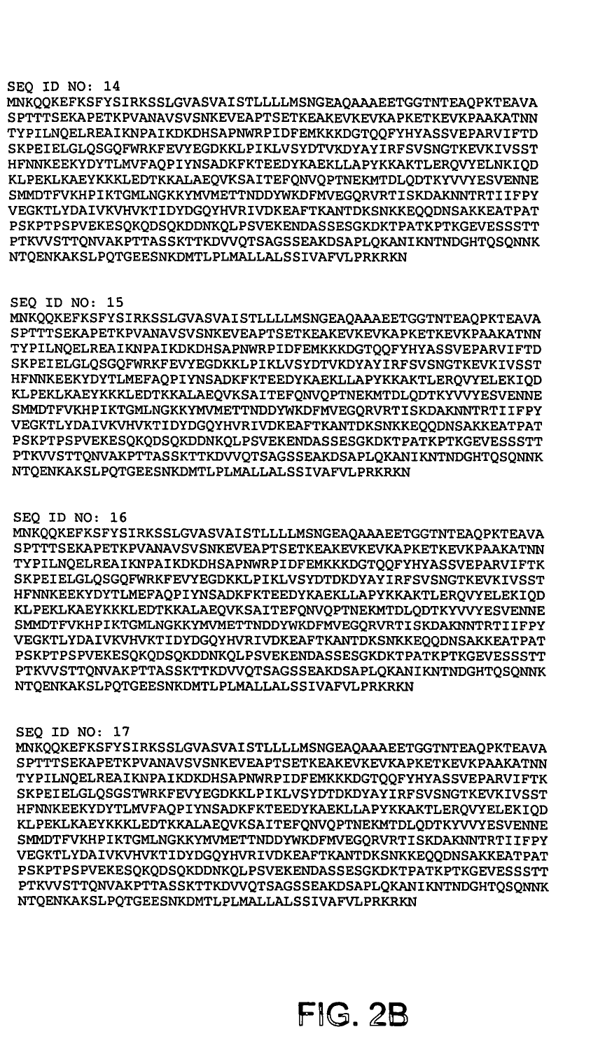

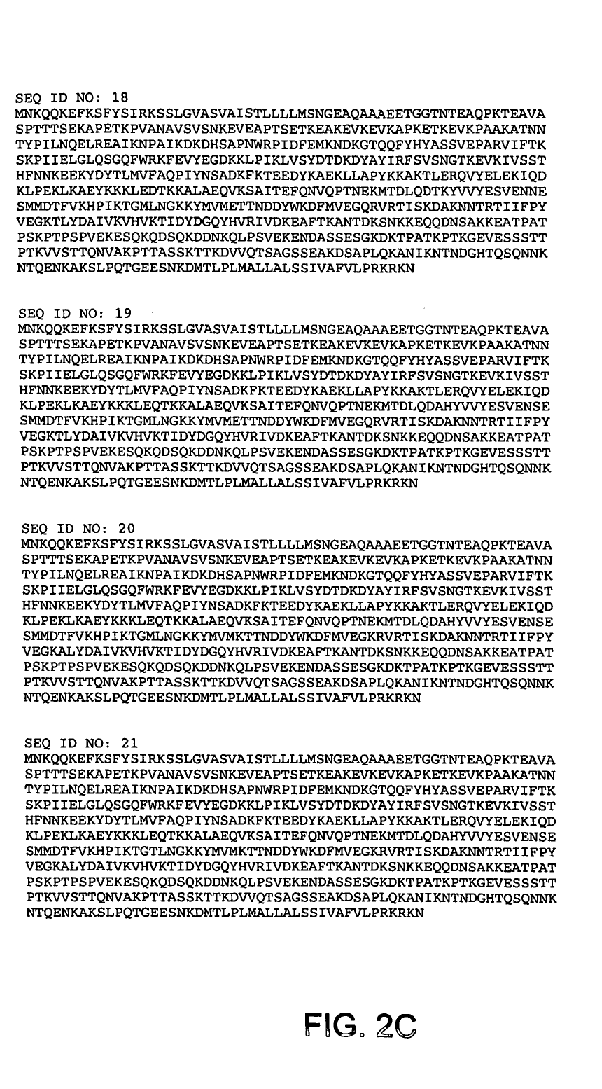

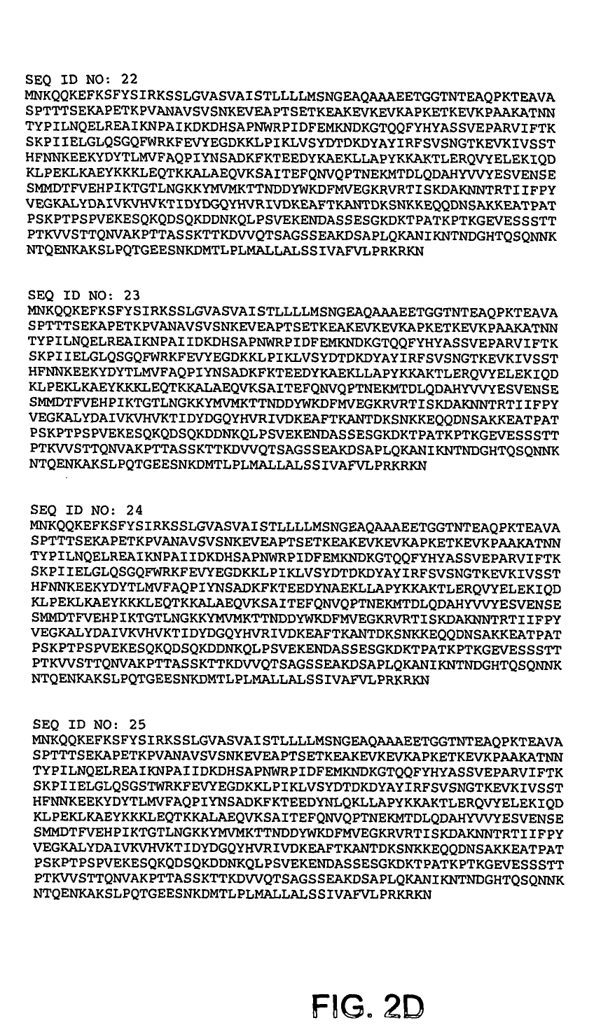

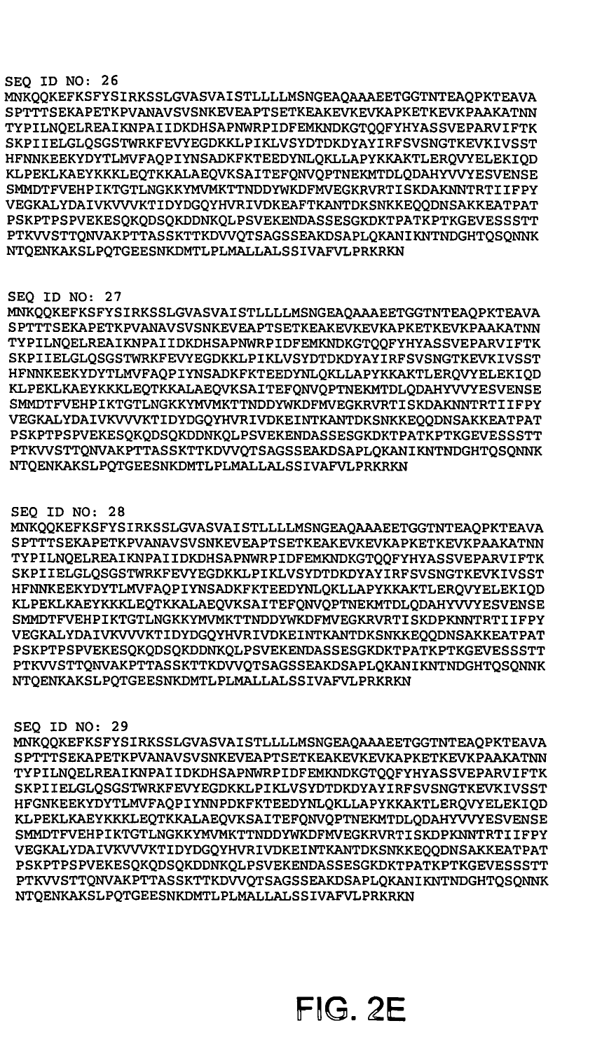

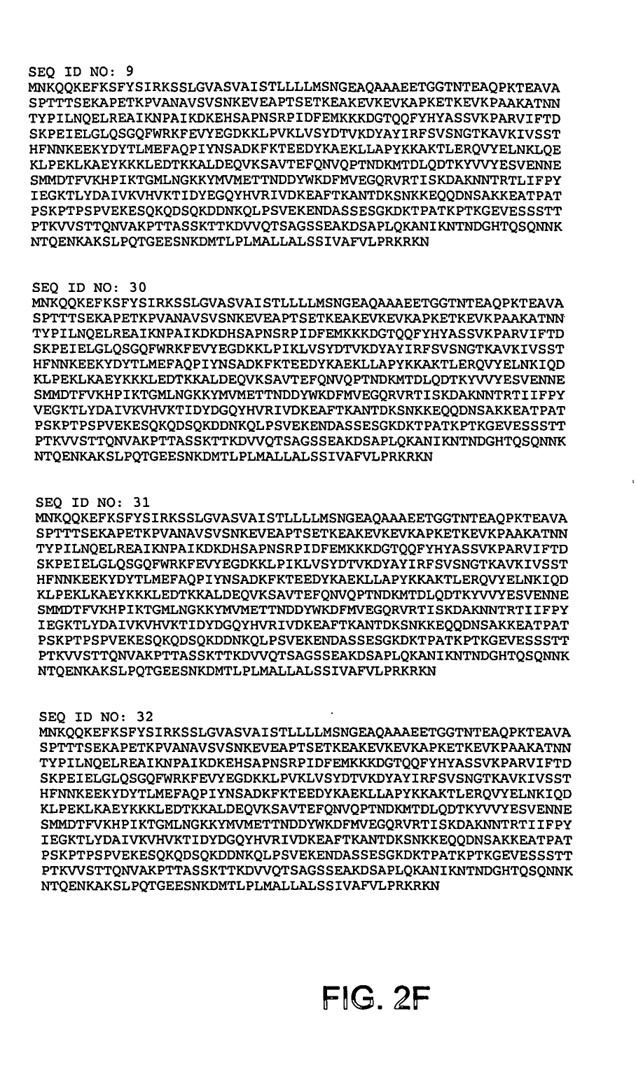

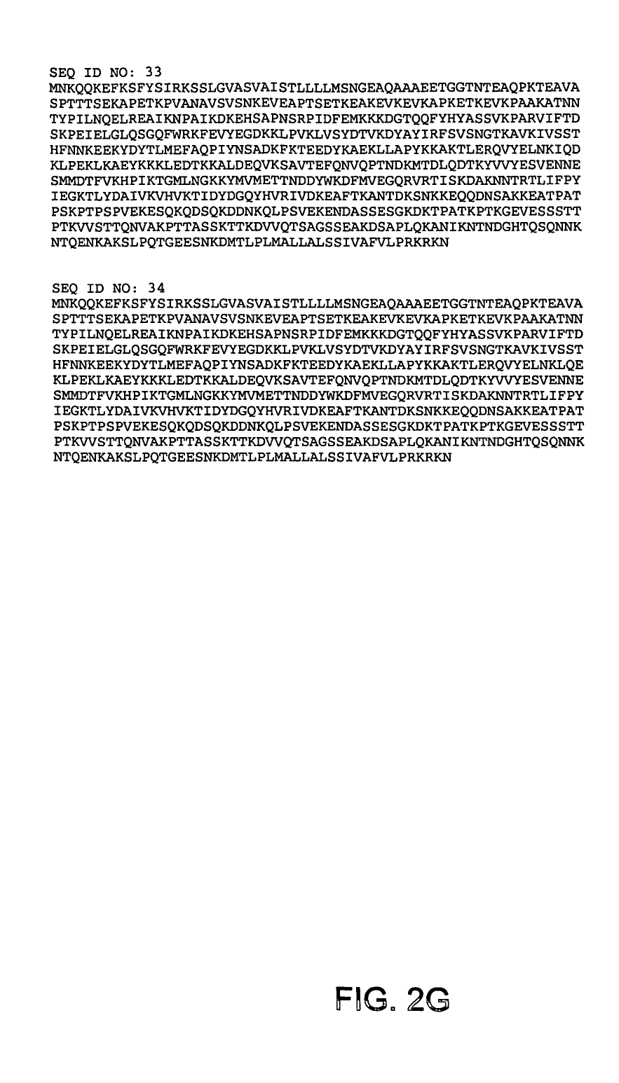

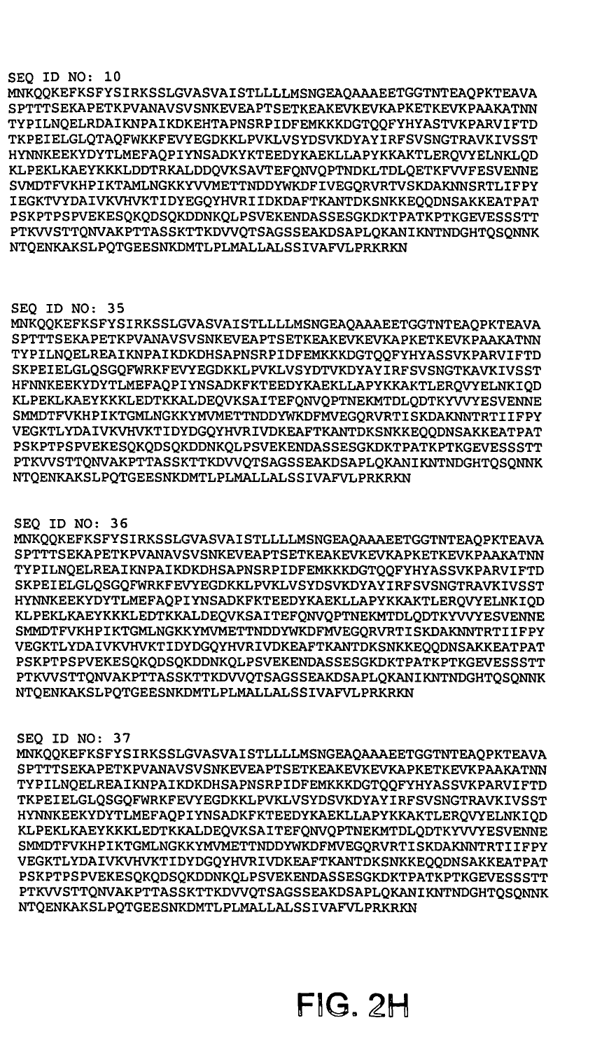

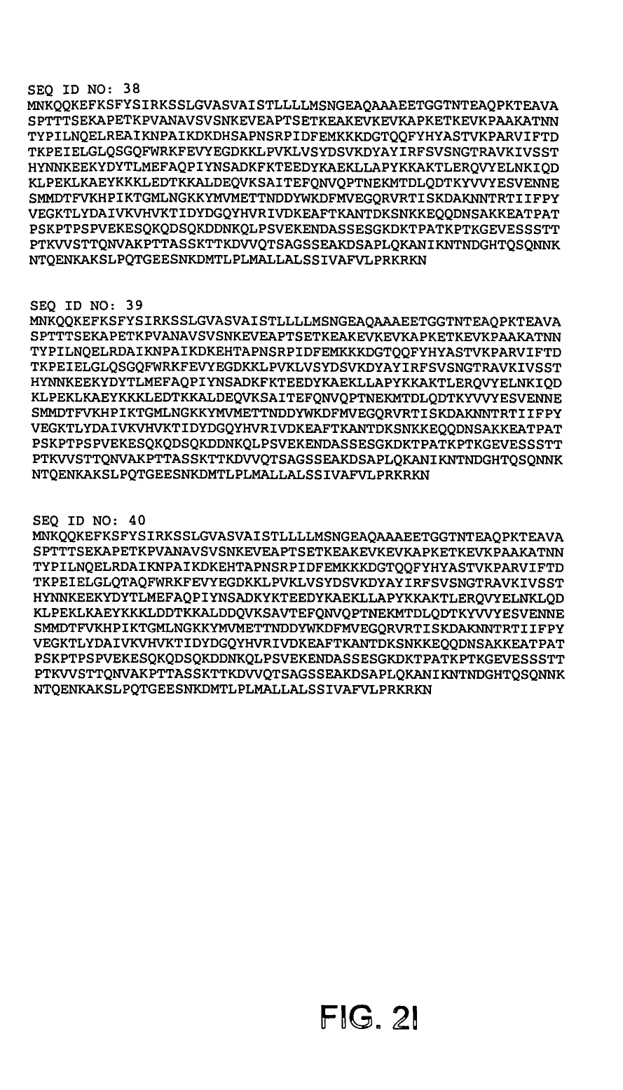

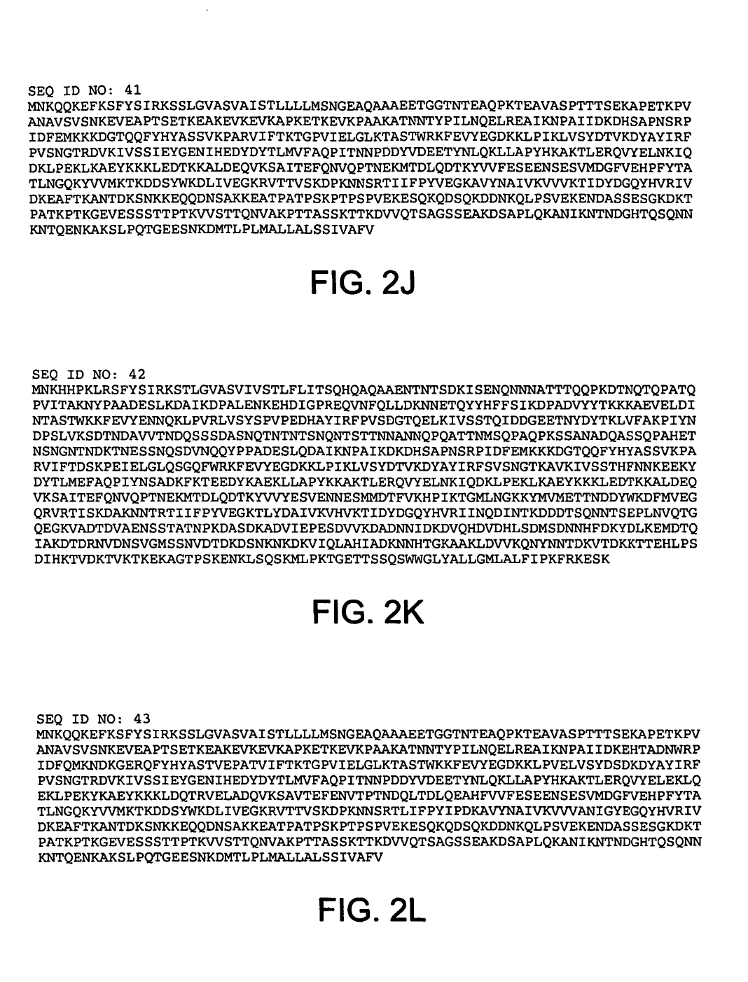

Additional hybrids may, for example, contain one or more modifications exemplified in SEQ ID NOs: 8, 9, and 10, or additional modifications that are apparent based on different amino acid sequences for an ORF0657n variant and ORF0190. Apparent modifications are those based on aligned amino acids differing between an ORF0657n and ORF0190, where an ORF0657n amino acid is changed to an ORF0190 amino acid. Examples of additional hybrid sequences include those provided by SEQ ID NOs: 11-43 (FIGS. 2A-2L).

Hybrid polypeptides may comprise modified ORF0657n sequence segments of different sizes. Preferably, the modified sequence is based on the ORF0657n central region spanning amino acids 122 to 448 or a fragment thereof. In different embodiments, the modified sequence segment is at least about 100, at least about 150, at least about 200, at least about 250, or at least about 300 amino acids in length.

Reference to “modified” or “altered” ORF0657n is a structural description taking into account the amino acid sequence of an ORF0657n polypeptide and ORF0190. A modified ORF0657n can be identified based on the presence of one or more stretches of at least 9 contiguous amino acids of a naturally occurring ORF0657n sequence. In different embodiments at least two, three, or four stretches of at least 9 contiguous amino acids of a naturally occurring ORF0657n sequence are present in the modified sequence segment.

Examples of naturally occurring ORF0657n sequences are provided by SEQ ID NOs: 2-7. Other naturally occurring sequences can be identified based on the presence of a high degree of sequence similarity or contiguous amino acids. Contiguous amino acids provide characteristic tags. In different embodiments, a naturally occurring ORF0657n sequence is a sequence found in a Staphylococcus, preferably S. aureus, having at least 20, at least 30, or at least 50 contiguous amino acids as in SEQ ID NO: 2; and/or having at least 75% sequence similarity or identity with SEQ ID NO: 2.

Sequence similarity and identity can be determined by different algorithms and techniques well known in the art. Generally, sequence similarity and identity is determined by aligning two sequences to obtain maximum amino acid identity between the two sequences, allowing for gaps, additions and substitutions in one of the sequences. Sequence similarity and identity can be determined based on the differences in the aligned sequence taking into account the overall length of the compared sequence.

Sequence identity can be determined by calculating the minimum number of amino acid alterations to an amino acid sequence required to arrive at a reference sequence divided by the number of amino acids in the reference sequence. Reference sequences for naturally occurring ORF0657n sequences provided herein are SEQ ID NOs: 2-7 and fragments thereof.

Reference sequences for naturally occurring ORF0657n can also be used to determine sequence similarity. Sequence similarity can be determined, for example, as indicated above using the program lalign (developed by Huang and Miller, Adv. Appl. Math. 12:337-357, 1991, for the <<sim>> program).

Different numbers of alterations may be present in a modified ORF0657n sequence segment. On the one hand, as the number of alterations increases similarity to ORF0190 more ORF0190 epitopes may be present. On the other hand, increasing the number of ORF0190 epitopes may decrease the number of ORF0657n epitopes.

Other factors that can be taken into account for an alteration include amino acid size, charge, polarity, and hydrophobicity. The effect of different amino acid side chains on properties of an amino acid are well known in the art. (See, for example, Ausubel, Current Protocols in Molecular Biology, John Wiley, 1987-2001, Appendix 1C.)

In exchanging amino acids to maintain activity, the replacement amino acid should have one or more similar properties such as approximately the same charge and/or size and/or polarity and/or hydrophobicity. For example, substituting valine for leucine, arginine for lysine, and asparagine for glutamine are good candidates for not causing a change in polypeptide functioning.

In different embodiments the minimum number of alterations is 8, 20, 25, 35, 45, or 55; and the maximum number of alterations, which may be present with any of the indicated minimum number of alterations having a lower number is 50, 60, 70, 79, 90, or 100. Each alteration is independently a deletion, addition, or substitution.

Amino acids can be represented by different designations as follows: A=Ala=Alanine: C=Cys=Cysteine: D=Asp=Aspartic acid: E=Glu=Glutamic acid: F=Phe=Phenylalanine: G=Gly=Glycine: H=His=Histidine: I=Ile=Isoleucine: K=Lys=Lysine: L=Leu=Leucine: M=Met=Methionine: N=Asn=Asparagine: P=Pro=Proline: Q=Gln=Glutamine: R=Arg=Arginine: S=Ser=Serine: T=Thr=Threonine: V=Val=Valine: W=Trp=Tryptophan: and Y=Tyr=Tyrosine.

In different embodiments the hybrid ORF0657n sequence segment comprises, consists, or consists essentially, of at least about 100 contiguous amino acids of the following Hybrid Structure:

X¹-AIKNPAI-X²-DK-X³-H-X⁴-APN-X⁵-RPIDFEMX^{-X}⁶-X⁷-
X⁸-G-X⁹-QQFYHYAS-X¹⁰-V-X¹¹-PARVIFT-X¹²-X¹³-K-X¹⁴-
IELGLQ-X¹⁵-X¹⁶-X¹⁷-X¹⁸-W-X¹⁹-KFEVYEGDKKLP-X²⁰-
KLVSYD-X²¹-X²²-KDYAYIRFSVSNGT-X²³-X²⁴-VKIVSSTH-
X²⁵-X²⁶-X²⁷-N-X²⁸-X²⁹-EKYDYTLM-X³⁰-FAQPIYN-X³¹-
X³²-DK-X³³-X³⁴-X³⁵-EEDY-X³⁶-X³⁷-X³⁸-
KLLAPYKKAKTLERQVYEL-X³⁹-K-X⁴⁰-Q-X⁴¹-
KLPEKLKAEYKKKL-X⁴²-X⁴³-T-X⁴⁴-KAL-X⁴⁵-X⁴⁶-QVKSA-
X⁴⁷-TEFQNV-X⁴⁸-PTN-X⁴⁹-K-X⁵⁰-TDLQ-X⁵¹-X⁵²-X⁵³-
X⁵⁴-VV-X⁵⁵-ESVEN-X⁵⁶-ES-X⁵⁷-MDTFV-X⁵⁸-HPIKT-X⁵⁹-
X⁶⁰-LNGKKY-X⁶¹-VM-X⁶²-TTND-X⁶³-YWKDF-X⁶⁴-VEG-
X⁶⁵-RVRT-X⁶⁶-SKD-X⁶⁷-KNN-X⁶⁸-RT-X⁶⁹-IFPY-X⁷⁰-
EGK-X⁷¹-X⁷²-YDAIVKV-X⁷³-VKTI-X⁷⁴-Y-X⁷⁵-GQYHVRI-
X⁷⁶-DK-X⁷⁷-X⁷⁸-X⁷⁹

where
X¹ is either E or a D alteration;
X² is either K or an I alteration;
X³ is either D or an E alteration;
X⁴ is either S or a T alteration;
X⁵ is either S or a W alteration;
X⁶ is either K or an N alteration;
X⁷ is either K or a D alteration;
X⁸ is either D or a K alteration;
X⁹ is either T or an E alteration;
X¹⁰ is either S or a T alteration;
X¹¹ is either K or an E alteration;
X¹² is either D or a K alteration;
X¹³ is either S or a T alteration;
X¹⁴ is either E or an I alteration;
X¹⁵ is either S or a T alteration;
X¹⁶ is either G or an A alteration;
X¹⁷ is either K or a S alteration;
X¹⁸ is either F or a T alteration;
X¹⁹ is either R or a K alteration;
X²⁰ is either I or a V alteration;
X²¹ is either T or an S alteration;
X²² is either V or a D alteration;
X²³ is either K or an R alteration;
X²⁴ is either A or an E alteration;
X²⁵ is either F or a Y alteration;
X²⁶ is either an optionally present G insertion alteration;
X²⁷ is either N or a E alteration;
X²⁸ is either K or a I alteration
X²⁹ is either E or a H alteration;
X³⁰ is either E or a V alteration;
X³¹ is either S or a N alteration;
X³² is either A or a P alteration;
X³³ is either F or an Y alteration;
X³⁴ is either K or a V alteration;
X³⁵ is either T or a D alteration;
X³⁶ is either K or a N alteration;
X³⁷ is either A or an L alteration;
X³⁸ is either E or a Q alteration;
X³⁹ is either N or an E alteration;
X⁴⁰ is either I or a L alteration;
X⁴¹ is either D or an E alteration;
X⁴² is either E or a D alteration;
X⁴³ is either D or a Q alteration;
X⁴⁴ is either K or an R alteration;
X⁴⁵ is either D or an A alteration;
X⁴⁶ is either E or a D alteration;
X⁴⁷ is either I or a V alteration;
X⁴⁸ is either Q or a T alteration;
X⁴⁹ is either E or a D alteration;
X⁵⁰ is either M or an L alteration;
X⁵¹ is either D or an E alteration;
X⁵² is either T or an A alteration
X⁵³ is either K or H alteration;
X⁵⁴ is either Y or an F alteration;
X⁵⁵ is either Y or an F alteration;
X⁵⁶ is either N or a S alteration;
X⁵⁷ is either M or a V alteration;
X⁵⁸ is either K or an E alteration;
X⁵⁹ is either G or an A alteration;
X⁶⁰ is either M or a T alteration;
X⁶¹ is either M or a V alteration;
X⁶² is either E or a K alteration;
X⁶³ is either D or a S alteration;
X⁶⁴ is either M or an I alteration;
X⁶⁵ is either Q or a K alteration;
X⁶⁶ is either I or a V alteration;
X⁶⁷ is either A or a P alteration;
X⁶⁸ is either T or an S alteration;
X⁶⁹ is either I or a L alteration;
X⁷⁰ is either V or an I alteration;
X⁷¹ is either T or an A alteration;
X⁷² is either L or a V alteration;
X⁷³ is either H or a V alteration;
X⁷⁴ is either D or a G alteration;
X⁷⁵ is either D or an E alteration;
X⁷⁶ is either V or an I alteration;
X⁷⁷ is either E or a D alteration;
X⁷⁸ is either A or an I alteration;
X⁷⁹ is either F or a N alteration; provided that at least 20 of the alterations are present.

With respect to the Hybrid Structure, in different embodiments the minimum number of alterations is 25, 35, 45, or 55; the maximum number of alterations, which may be present with any of the indicated minimum number of alterations having a lower number is 50, 60, 70, or 79; and/or one or more of the following combinations of alterations are present:
X⁶-X⁷-X⁸ is either KKD or NDK alterations;
X¹⁷-X¹⁸ is either KF or ST alterations;
X²⁶-X²⁷ is either N or GE alterations;
X²⁸-X²⁹ is either KE or IH alterations;
X³¹-X³² is either SA or NP alterations;
X³⁴-X³⁵ is either KT or VD alterations;
X³⁶-X³⁷-X³⁸ is either KAE or NLQ alterations; and
X⁵²-X⁵³ is either TK or AH alterations.

Hybrid polypeptides may contain additional amino acid regions. Such regions should not prevent the hybrid polypeptide from providing ORF0190 and ORF0657n epitopes. Additional regions can be based on ORF0190, ORF0657n or other amino acid sequences.

Preferably, additional regions if present provide a useful purpose such as providing epitopes from other bacterial polypeptides, providing an affinity tag to facilitate polypeptide purification, enhancing polypeptide efficacy, or enhancing polypeptide stability. Polypeptide production can, for example, be facilitated through the use of an initiation codon (e.g., coding for methionine) suitable for recombinant expression, and the introduction of restriction enzyme recognition sites.

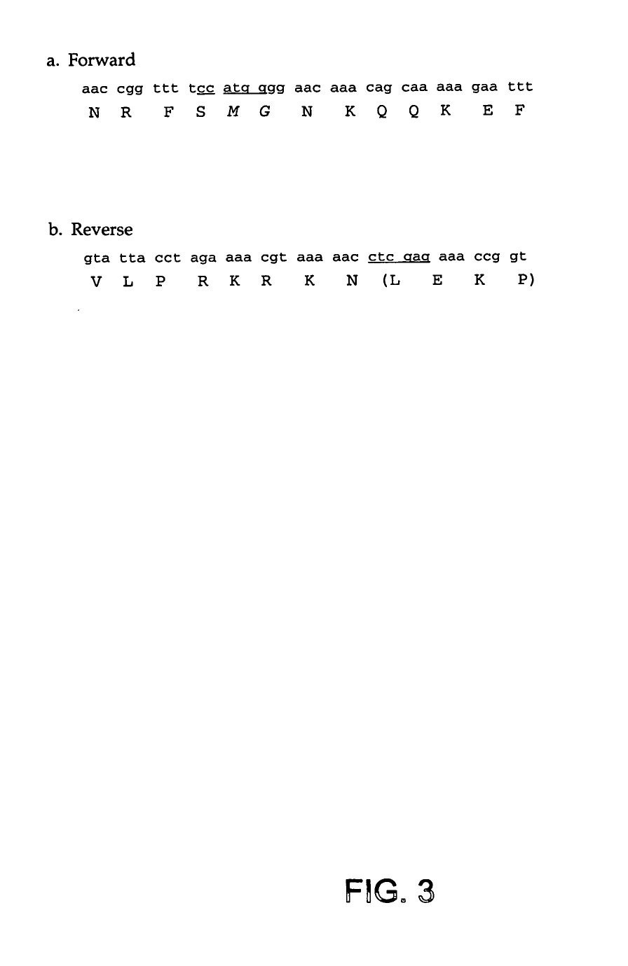

The introduction of a restriction site can be illustrated by Example 1 provided infra and FIG. 3. For example, using the forward primer provided in FIG. 3, a restriction recognition site can be introduced into a hybrid or 0657n polypeptide. The illustrated restriction site is accompanied by a glycine addition after methionine.

Efficacy of a polypeptide to induce an immune response can be enhanced through epitope enhancement. Epitope enhancement can be performed using different techniques such as those involving alteration of anchor residues to improve peptide affinity for MHC molecules and those increasing affinity of the peptide-MHC complex for a T-cell receptor. (Berzofsky et al., 2001. Nature Review 1:209-219.)

Polypeptide purification can be enhanced by adding a group to the carboxy or N-terminus to facilitate purification. Examples of groups that can be used to facilitate purification include polypeptides providing affinity tags. Examples of affinity tags include a six-histidine tag, trpE, glutathione and maltose-binding protein.

The ability of a polypeptide to produce an immune response can be enhanced using groups that generally enhance an immune response. Examples of groups that can be joined to a polypeptide to enhance an immune response against the polypeptide include cytokines such as IL-2. (Buchan et al., 2000. Molecular Immunology 37:545-552.)

ORF0657n Sequences

ORF0657n has been implicated to have a role in S. aureus iron acquisition. (Andrade et al., Genome Biology 3(9):47.1-47.5, 2003.) ORP0657n sequences, some of which are from different sources, have been given different designations in different references. (For example, see, Etz et al., PNAS USA, 99:6573-6578, 2002 (LPXTGVI); Baba et al., The Lancet 359:1819-1827, 2002 (MW1011); Kuroda, et al., The Lancet 357, 1225-1240, 2001 (SA0976); Andrade et al., Genome Biology 3(9):47.1-47.5, 2003 (S_aur2); Mazmanian et al., Science 299:906-909, 2003 (isdB); Mazmanian et al., Molecular Microbiology 40:1049-1057, 2001 (sasJ); and Taylor et al., Mol. Microbiol. 43:1603-1614, 2002 (sirH).

A polypeptide sequence corresponding to a ORF0657n protein sequence appears to be provided in different patent publications. (Meinke et al., International Publication Number WO 02/059148, published Aug. 1, 2002, Wang et al., International Publication Number WO 02/077183, published Oct. 3, 2002, Masignani et al., International Publication Number WO 02/094868, published Nov. 28, 2002, Foster et al., International Publication Number WO 02/102829, published Dec. 27, 2002, and Foster et al., International Publication Number WO 03/011899, published Feb. 13, 2003.)

Additional examples of S. aureus ORF0657n protein sequences are provided by SEQ ID NOs: 3-7. S. aureus ORF0657n cDNA encoding SEQ ID NOs: 3-7 are provided by SEQ ID NOs: 52-56.

Immunogens

Immunogens containing a hybrid polypeptide also contain one or more additional regions or moieties joined to the polypeptide. The additional regions may be polypeptide regions or may be regions that are not polypeptides. Additional regions and moieties, if present, are preferably covalently joined to the carboxy or amino terminus of the hybrid polypeptide.

An additional region or moiety that is present should not significantly prevent a hybrid polypeptide from providing S. aureus epitopes that can be used for diagnostic or therapeutic purposes. Preferably, an additional region or moiety is present to achieve a particular purpose, such as to enhance polypeptide stability, purification, or the ability to produce an immune response.

Polypeptide stability can be enhanced by modifying the carboxy or N-terminus. Examples of possible modifications include amino terminus protecting groups such as acetyl, succinyl, benzyl, benzyloxycarbonyl or t-butyloxycarbonyl; carboxy terminus protecting groups such as amide, methylamide, and ethylamide; and groups such as polyethylene glycol that may be present on the amino or carboxy terminus.

Polypeptide Production

Polypeptides can be produced using standard techniques including those involving chemical synthesis and those involving purification from a cell producing the polypeptide. Techniques for chemical synthesis of polypeptides are well known in the art. (See e.g., Vincent, Peptide and Protein Drug Delivery, New York, N.Y., Decker, 1990.)

Polypeptides can be purified from a cell using techniques well known in the art. (See for example, Ausubel, Current Protocols in Molecular Biology, John Wiley, 1987-1998.) Obtaining polypeptides from a cell is facilitated using recombinant nucleic acid techniques to produce the polypeptide. Recombinant nucleic acid techniques for producing a polypeptide involve introducing, or producing, a recombinant gene encoding the polypeptide in a cell and expressing the polypeptide.

Recombinant nucleic acid is nucleic acid that by virtue of its sequence or form does not occur in nature. Possible forms for recombinant nucleic acid include isolation from nucleic acid found in a cell; or a combination of nucleic acid sequences not found in nature.

A recombinant gene contains recombinant nucleic acid encoding a polypeptide along with regulatory elements for proper transcription and processing. The encoding nucleic acid is in a form different than naturally occurring nucleic acid encoding the polypeptide. Differences in form include separation from other nucleic acid naturally associated with the encoding nucleic acid or present in a combination with other nucleic acid not naturally associated with the encoding nucleic acid. The recombinant gene can be present in a cellular genome or can be part of an extrachromosomal element or vector.

The regulatory elements that may be present as part of a recombinant gene include those naturally associated with the polypeptide encoding sequence and exogenous regulatory elements not naturally associated with the polypeptide encoding sequence. Exogenous regulatory elements such as an exogenous promoter can be useful for expressing a recombinant gene in a particular host, or increasing the level of expression. Generally, the regulatory elements that are present in a recombinant gene include a transcriptional promoter, a ribosome binding site, a terminator, and an optionally present operator. A preferred element for processing in eukaryotic cells is a polyadenylation signal.

Expression of a recombinant gene in a cell is facilitated through the use of an expression vector. Preferably, an expression vector in addition to a recombinant gene also contains an origin of replication for autonomous replication in a host cell, a selectable marker, useful restriction enzyme sites, and a potential for high copy number. Examples of expression vectors are cloning vectors, modified cloning vectors, specifically designed plasmids and viruses.

Due to the degeneracy of the genetic code, a large number of different encoding nucleic acid sequences can be used to code for a particular polypeptide. The degeneracy of the genetic code arises because almost all amino acids are encoded by different combinations of nucleotide triplets or “codons”. Amino acids are encoded by codons as follows:
A=Ala=Alanine: codons GCA, GCC, GCG, GCU
C=Cys=Cysteine: codons UGC, UGU
D=Asp=Aspartic acid: codons GAC, GAU
E=Glu=Glutamic acid: codons GAA, GAG
F=Phe=Phenylalanine: codons UUC, UUU
G=Gly=Glycine: codons GGA, GGC, GGG, GGU
H=His=Histidine: codons CAC, CAU
I=Ile=Isoleucine: codons AUA, AUC, AUU
K=Lys=Lysine: codons AAA, AAG
L=Leu=leucine: codons UUA, UUG, CUA, CUC, CUG, CUU
M=Met=Methionine: codon AUG
N=Asn=Asparagine: codons AAC, AAU
P=Pro=Proline: codons CCA, CCC, CCG, CCU
Q=Gln=Glutamine: codons CAA, CAG
R=Arg=Arginine: codons AGA, AGG, CGA, CGC, CGG, CGU
S=Ser=Serine: codons AGC, AGU, UCA, UCC, UCG, UCU
T=Thr=Threonine: codons ACA, ACC, ACG, ACU
V=Val=Valine: codons GUA, GUC, GUG, GUU
W=Trp=Tryptophan: codon UGG
Y=Tyr=Tyrosine: codons UAC, UAU

Suitable cells for recombinant nucleic acid expression polypeptides are prokaryotes and eukaryotes. Examples of prokaryotic cells that can be employed include E. coli; members of the Staphylococcus genus, such as S. aureus; members of the Lactobacillus genus, such as L. plantarum; members of the Lactococcus genus, such as L. lactis; and members of the Bacillus genus, such as B. subtilis. Examples of eukaryotic cells that can be employed include mammalian cells; insect cells; yeast cells such as members of the Saccharomyces genus (e.g., S. cerevisia) and members of the Pichia genus (e.g., P. pastoris).

Techniques for recombinant gene production, introduction into a cell, and recombinant gene expression are well known in the art. Examples of such techniques are provided in references such as Ausubel, Current Protocols in Molecular Biology, John Wiley, 1987-2002, and Sambrook et al., Molecular Cloning, A Laboratory Manual, 2^{nd} Edition, Cold Spring Harbor Laboratory Press, 1989.

If desired, expression in a particular host can be enhanced through codon optimization. Codon optimization includes use of more preferred codons. Techniques for codon optimization in different hosts are well known in the art.

Depending upon the host used for expression, polypeptides may contain post translational modifications. Reference to “polypeptide” or an “amino acid” sequence of a polypeptide includes polypeptides containing one or more amino acids having a structure of a post-translational modification from a host cell, such as a yeast host.

Polypeptides may contain post translational modifications, for example, N-linked glycosylation, O-linked glycosylation, or acetylation. Reference to “polypeptide” or an “amino acid” sequence of a polypeptide includes polypeptides containing one or more amino acids having a structure of a post-translational modification from a host cell, such as a yeast host.

Post translational modifications can be produced chemically or by making use of suitable hosts. For example, in S. cerevisiae the nature of the penultimate amino acid appears to determine whether the N-terminal methionine is removed. Furthermore, the nature of the penultimate amino acid also determines whether the N-terminal amino acid is N^{α}-acetylated (Huang et al., Biochemistry 26: 8242-8246, 1987). Another example includes a polypeptide targeted for secretion due to the presence of a secretory leader (e.g., signal peptide), where protein is modified by N-linked or O-linked glycosylation. (Kukuruzinska et al., Ann. Rev. Biochem. 56:915-944, 1987.)

Adjuvants

Adjuvants are substances that can assist an immunogen in producing an immune response. Adjuvants can function by different mechanisms such as one or more of the following: increasing the antigen biologic or immunologic half-life; improving antigen delivery to antigen-presenting cells; improving antigen processing and presentation by antigen-presenting cells; and inducing production of immunomodulatory cytokines. (Vogel, Clinical Infectious Diseases 30(suppl. 3):S266-270, 2000.)

A variety of different types of adjuvants can be employed to assist in the production of an immune response. Examples of particular adjuvants include aluminum hydroxide, aluminum phosphate, other salts of aluminum, calcium phosphate, DNA CpG motifs, monophosphoryl lipid A, cholera toxin, E. coli heat-labile toxin, pertussis toxin, muramyl dipeptide, Freund's incomplete adjuvant, MF59, SAF, immunostimulatory complexes, liposomes, biodegradable microspheres, saponins, nonionic block copolymers, muramyl peptide analogues, polyphsophazene, synthetic polynucleotides, IFN-γ, IL-2 and IL-12. (Vogel Clinical Infectious Diseases 30(suppl 3):S266-270, 2000, Klein et al., Journal of Pharmaceutical Sciences 89, 311-321, 2000.)

Patients For Inducing Protective Immunity

A “patient” refers to a mammal capable of being infected with S. aureus. A patient can be treated prophylactically or therapeutically. Prophylactic treatment provides sufficient protective immunity to reduce the likelihood, or severity, of a S. aureus infection. Therapeutic treatment can be performed to reduce the severity of a S. aureus infection.

Prophylactic treatment can be performed using a vaccine containing an immunogen described herein. Such treatment is preferably performed on a human. Vaccines can be administered to the general population or to those persons at an increased risk of S. aureus infection.

Persons with an increased risk of S. aureus infection include health care workers; hospital patients; patients with a weakened immune system; patients undergoing surgery; patients receiving foreign body implants, such a catheter or a vascular device; patients facing therapy leading to a weakened immunity; and persons in professions having an increased risk of burn or wound injury. (The Staphylococci in Human Disease, Crossley and Archer (ed.), Churchill Livingstone Inc. 1997.)

Non-human patients that can be infected with S. aureus include horses, cows, pigs, sheep, goats, rabbits, horses, dogs, cats and mice. Treatment of non-human patients is useful in protecting pets and livestock, and in evaluating the efficacy of a particular treatment.

Combination Vaccines

Hybrid polypeptides can be used alone, or in combination with other immunogens, to induce an immune response. Additional immunogens that may be present include: one or more additional S. aureus immunogens, such as those referenced in the Background of the Invention supra; one or more immunogens targeting one or more other Staphylococcus organisms such as S. epidemmidis, S. haemolyticus, S. warneri, or S. lugunensis; and one or more immunogens targeting other infections organisms.

Animal Model System

An animal model system was developed to evaluate the efficacy of an immunogen to produce a protective immune response against S. aureus. Two obstacles encountered in setting up a protective animal model were: (1) very high challenge dose needed to overcome innate immunity and (2) death rate too fast to detect a protective response. Specifically, after bacterial challenge mice succumbed to infection within 24 hours which did not provide sufficient time for the specific immune responses to resolve the infection. If the dose was lowered both control and immunized mice survived the infection.

These obstacles were addressed by developing a slow kinetics lethality model involving S. aureus prepared from cells in stationary phase, appropriately titrated, and intravenously administered. This slow kinetics of death provides sufficient time for the specific immune defense to fight off the bacterial infection (e.g., 10 days rather 24 hours).

Staphylococcus cells in stationary phase can be obtained from cells grown on solid medium. They can also be obtained from liquid, however the results with cells grown on solid media were more reproducible. Cells can conveniently be grown overnight on solid medium. For example, S. aureus can be grown from about 18 to about 24 hours under conditions where the doubling time is about 20-30 minutes.

Staphylococcus can be isolated from solid or liquid medium using standard techniques to maintain Staphylococcus potency. Isolated Staphylococcus can be stored, for example, at −70° C. as a washed high density suspension (>10⁹ colony forming units (CFU)/mL) in phosphate buffered saline containing glycerol.

The S. aureus challenge should have a potency providing about 80 to 90% death in an animal model over a period of about 7 to 10 days starting on the first or second day. Titration experiments can be performed using animal models to monitor the potency of the stored Staphylococcus inoculum. The titration experiments can be performed about one to two weeks prior to an inoculation experiment.

Initial potency for titration experiments can be based on previous experiments. For S. aureus and the animal model strain Becker a suitable potency was generally found in the range of 5×10⁸ to 8×10⁸ CFU/ml.

Different types of Staphylococcus can be evaluated in an animal model, such as S. aureus, S. epidermidis, S. haemolyticus, S. warneri, or S. lugunensis. In a preferred embodiment the Staphylococcus is S. aureus.

Administration

Immunogens can be formulated and administered to a patient using the guidance provided herein along with techniques well known in the art. Guidelines for pharmaceutical administration in general are provided in, for example, Vaccines Eds. Plotkin and Orenstein, W. B. Sanders Company, 1999; Remington's Pharmaceutical Sciences 20^{th} Edition, Ed. Gennaro, Mack Publishing, 2000; and Modern Pharmaceutics 2^{nd} Edition, Eds. Banker and Rhodes, Marcel Dekker, Inc., 1990, each of which are hereby incorporated by reference herein.

Pharmaceutically acceptable carriers facilitate storage and administration of an immunogen to a patient. Pharmaceutically acceptable carriers may contain different components such as a buffer, sterile water for injection, normal saline or phosphate buffered saline, sucrose, histidine, salts and polysorbate.

Immunogens can be administered by different routes such as subcutaneous, intramuscular, or mucosal. Subcutaneous and intramuscular administration can be performed using, for example, needles or jet-injectors.

Suitable dosing regimens are preferably determined taking into account factors well known in the art including age, weight, sex and medical condition of the patient; the route of administration; the desired effect; and the particular compound employed. The immunogen can be used in multi-dose vaccine formats. It is expected that a dose would consist of the range of 1 μg to 1.0 mg total polypeptide, in an embodiment of the present invention the range is 0.1 mg to 1.0 mg.

The timing of doses depends upon factors well known in the art. After the initial administration one or more booster doses may subsequently be administered to maintain or boost antibody titers. An example of a dosing regime would be day 1, 1 month, a third dose at either 4, 6 or 12 months, and additional booster doses at distant times as needed.

Generation of Antibodies

A hybrid polypeptide can be used to generate antibodies and antibody fragments that bind to the polypeptide or to S. aureus. Such antibodies and antibody fragments have different uses including use in polypeptide purification, S. aureus identification, or in therapeutic or prophylactic treatment against S. aureus infection.

Antibodies can be polyclonal or monoclonal. Techniques for producing and using antibodies are well known in the art. Examples of such techniques are described in Ausubel, Current Protocols in Molecular Biology, John Wiley, 1987-1998, Harlow et al., Antibodies, A Laboratory Manual, Cold Spring Harbor Laboratory, 1988, and Kohler et al., Nature 256:495-497, 1975.

### EXAMPLES

Examples are provided below further illustrating different features of the present invention. The examples also illustrate useful methodology for practicing the invention. These examples do not limit the claimed invention.

### Example 1

### Use of ORF0657n to Provide Protective Immunity

This example illustrates the ability of ORF0657n to provide protective immunity in a model.

Mutated ORF0657n Cloning and Expression

An ORF0657n DNA sequence (SEQ ID NO: 45) was translated using Vector NTI software and the resulting 645 amino acid sequence (SEQ ID NO: 2) was analyzed. PCR primers were designed to amplify the gene starting at the first asparagine residue and ending prior to the stop codon at the terminal asparagine residue (FIG. 3). These PCR primers also had additional NcoI (forward primer) and XhoI (reverse primer) sites to facilitate cloning into the expression vector.

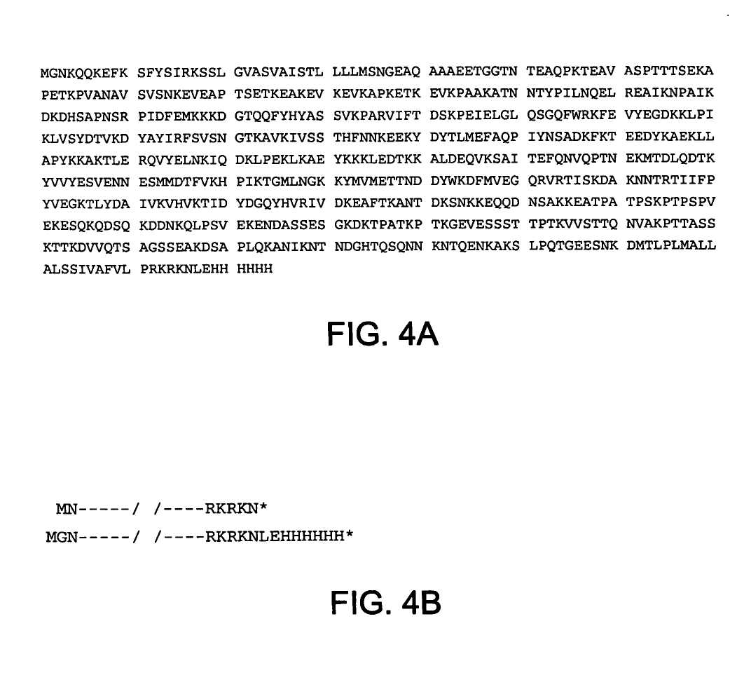

The protein was designed to be expressed from the pET28 vector with the terminal His residues and the stop codon encoded by the vector. In addition, a glycine residue was added to the protein after the methionine initiator. The resulting amplified (1964 bp) DNA sequence encodes a form of mature ORF0657n (FIG. 4a). FIG. 4B shows an alignment of the translation of the native ORF0657n with that of the expressed construction.

PCR amplified sequences were ligated into the pET28 vector (Novagen) using the NcoI/XhoI sites that had been engineered into the PCR primers and introduced into E. coli DH5α (Invitrogen) by heat shock. Colonies were selected, grown in LB with 30 μg/mL kanamycin, DNA minipreps made (Promega), and insert integrity determined by restriction digestion and PCR. Four minipreps with correct insert size were sequenced using the primers listed in Table 1. A clone was selected containing no DNA changes from the desired sequence.

TABLE 1

| | | |
|---|---|---|
| SEQ ID NO: | Description | Sequence |

| | | |
|---|---|---|
| 50 | M13F | 5′-CTGGCCGTCGTTTTAC |
| 51 | M13R | 5′-CAGGAAACAGCTATGAC |
| 46 | ORF0657nF | 5′-AACCGGTTTTCCATGGGGAACAAA |
| | | CAGCAAAAAGAATTT-3′ |
| 48 | ORF0657nR | 5′-ACCGGTTTCTCGAGTTAGTTTTTA |
| | | CGTTTTCTAGGTAATAC-3′ |

E. coli HMS174(DE3) cells (Novagen) were transformed and grown on LB plates containing kanamycin (30 ug/ml); 3 colonies (UnkC-1, UnkC-2 and UnkC-3) were selected for expression testing. Liquid LB (kanamycin) cultures were incubated at 37° C., 250 rpm until the A₆₀₀ was between 0.6 and 1.0 and then induced by the addition of IPTG to final concentrations of 1 mM followed by three hours further incubation. Cultures were harvested by centrifugation at 5000×g for 5 minutes at 4° C. Cells were resuspended in 500 μl lysis buffer (Bug Buster, with protease inhibitors, Novagen). An equal volume of loading buffer (supplemented with β-mecapto ethanol to 5% final volume) was added prior to heating the samples at 70° C. for 5 minutes. Extracts were run on Novex 4-20% Tris-Glycine gels and assayed for protein (Coomassie Blue stained) and blotted onto nitrocellulose and probed with anti-HIS6 antibodies (Zymedd).

Mutated ORF0657n Purification

Direct scale-up of the above small scale procedure into stirred tank fermenters (75 liter scale) with a 50 liter working volume was achieved. Inoculum was cultivated in a 250 mL flask containing 50 mL of Luria-Bertani (LB) medium (plus Kanamycin) and inoculated with 1 mL of frozen seed culture and cultivated for 3 hours. One mL of this seed was used to inoculate a 2 liter flask containing 500 mL of LB medium (plus Kanamycin) and incubated for 16 hours. A large scale fermenter (75 liter scale) was cultivated with 50 liters of LB medium (plus Kanamycin). The fermentation parameters of the fermenter were: pressure=5 psig, agitation speed=300 RPMs, airflow=15 liters/minute and temperature=37° C. Cells were incubated to an optical density (OD) of 0.8 optical density units, at a wavelength of 600 nm, and induced with Isopropyl-B-K-Thiogalactoside (IPTG) at a concentration of 1 mM. Induction time, with IPTG, was three hours. Cells were harvested by lowering the temperature to 15° C., concentration through a 500KMWCO hollow fiber cartridge, and centrifuged at 9,000 times gravity at 4° C. for 20 minutes. Supernates were decanted and the recombinant E. coli wet cell pellets were frozen at −70° C.

Recombinant E. coli cells (19.2 grams wet cell weight) were suspended in Lysis Buffer (50 mM Tris-HCl, pH 8.0, 0.1 M NaCl, 2 mM MgCl₂, 10 mM imidazole, 0.1% Tween™-80, and 6 M guanidine-HCl) at 8 ml per gram of cell wet weight. Protease Inhibitor Cocktail for use with poly-(Histidine)-tagged proteins (Sigma, P8849) was added to the suspension at 0.05 ml per gram of cell paste. Additionally, Lysozyme was added to 1 mg/mL, and Benzonase™ (EM Ind.) was added to 1 μL/mL. Cell lysis was accomplished by passing the suspension through a microfluidizer at 14,000 PSI (Microfluidics Model 110S) four times at 4° C. Cell debris was pelleted at 11,000×g for 30 minutes at 4° C., and the supernatant retained.

Proteins bearing a His-tag were purified from the supernatant. The supernatant was mixed with 20 mL of Ni⁺-NTA agarose (Qiagen) at 4° C. with gentle inversion for 2 hours. The mixture was poured into an open column (1.5 cm×20 cm) and the non-bound fraction was collected in bulk. The column was washed with Wash Buffer (20 mM Tris-HCl, pH 8.0, 0.15 M NaCl, 0.1% Tween™-80). His-tagged ORF0657n was eluted with a step gradient of 300 mM imidazole, 20 mM Tris-HCl, pH 7.5, 0.15 M NaCl, 0.1% Tween™-80.

Fractions containing mutated ORF0657n were detected by Coomassie stained SDS-PAGE and pooled. Pooled fractions were filtered through a 0.2 micron filter to remove particulate material, and were applied on a size-exclusion column (Sephacryl S-300 26/60 column, Amersham Biosciences) and eluted at 1 mL/min with 10 mM MOPS pH 7.1, 150 mM NaCl. Fractions containing mutated ORF0657n were detected by Coomassie stained SDS-PAGE and Western blotting (anti-tetra His Mab, Qiagen). Endotoxin was removed by filtration through a Zeta-Plus™ Biofilter (CUNO). Protein was determined by BCA (Pierce). Purity was determined by densitometry of Coomassie stained gels.

Preparation of S. Aureus Challenge

S. aureus was grown on TSA plates at 37° C. overnight. The bacteria were washed from the TSA plates by adding 5 ml of PBS onto a plate and gently resuspending the bacteria with a sterile spreader. The bacterial suspension was spun at 6000 rpm for 20 minutes using a Sorvall RC-5B centrifuge (DuPont Instruments). The pellet was resuspended in 16% glycerol and aliquots were stored frozen at −70° C.

Prior to use, inocula were thawed, appropriately diluted and used for infection. Each stock was titrated at least 3 times to determine the appropriate dose inducing slow kinetics of death in naive mice. The potency of the bacterial inoculum (80 to 90% lethality) was constantly monitored to assure reproducibility of the model. Ten days before each challenge experiment, a group of 10 control animals (immunized with adjuvant alone) were challenged and monitored.

Protection Studies for Mutated ORF0657n

Twenty-five BALB/c mice were immunized with three doses of mutated ORF0657n (20 μg per dose) on aluminum hydroxyphosphate adjuvant (450 μg per dose). Aluminum hydroxyphosphate adjuvant (AHP) is described by Klein et al., Journal of Pharmaceutical Sciences 89, 311-321, 2000. The doses were administered as two 50 μl intramuscular injections on days 0, 7 and 21. The mice were bled on day 28, and their sera were screened by ELSIA for reactivity to mutated ORF0657n.

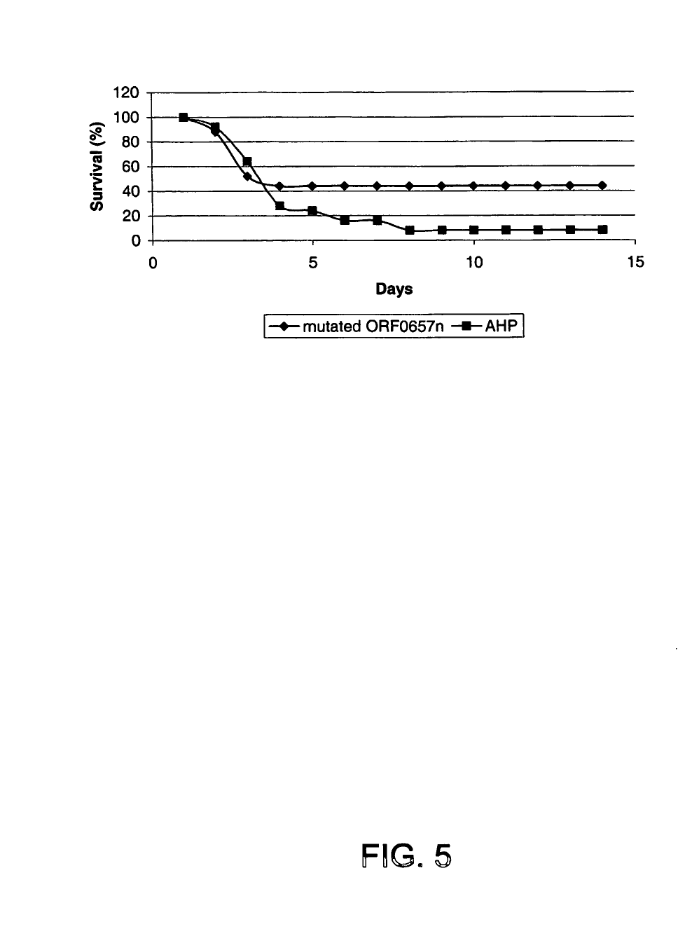

On day 35 of the experiment the mice were challenged by intravenous injection of S. aureus grown at a dose (7.3×10⁸ CFU ml) determined in titration experiments to cause death over a period of approximately 2 to 7 days. Survival in this lethal model with slow kinetics of death was evaluated against a control set of mice that had just been sham-immunized with AHP. The mice were monitored over a 14 day period for survival (FIG. 5). At the end of the experiment 11 mice survived the ORF0657n immunized group compared to three surviving in the AHP control group.

### Example 2

### Obtaining ORF0657n Sequences

Different S. aureus clinical isolates were added to 3 ml of Difco Tryptic Soy Broth (Becton Dickinson, Sparks, Md.) and incubated overnight at 37° C. and 150 rpm. The overnight cultures were centrifuged in 1.5 ml Eppendorf tubes at 14,000 rpm for 5 minutes. The broth was decanted and the pellets re-suspended in 5001 μl re-suspension buffer (25% sucrose, 10 mM Tris pH 7.5). A 5 μl of a 2 mg/ml lysostaphin (Sigma-Aldrich, St. Louis, Mo.) solution was added to each resuspended pellet. Suspensions were then incubated at 37° C. for 1 hour. At the end of the incubation period, 250 μl of 2% SDS was added to each tube and vortexed until the viscosity of the solution noticeably decreased. 250 μl phenol-chloroform-isoamyl solution (25:24:1, v/v) (Gibco/Invitrogen Corporation, Grand Island, N.Y.) were added. The mixture was vortexed for 30 seconds and centrifuged for 5 minutes at 14,000 rpm. The top aqueous phase was removed and the precipitation steps were repeated until barely any interface remained. 0.1 volume of 3 M NaOAc, pH 4.8, was added to each tube and mixed. One volume of isopropanol was then added and mixed again. The tubes were left to incubate 5 minutes at room temperature and then centrifuged at 14,000 rpm for 15 minutes. The supernatant was decanted and tubes were allowed to dry upside-down on tissue. The pellets were resuspended in 50 μl sterile H₂O.

The isolated DNA was used as a template for PCR. The gene was amplified using the following PCR primers: forward primer (SEQ ID NO: 46) and reverse primer (SEQ ID NO: 48). PCR products were sequenced using standard Big Dye protocols.

Other embodiments are within the following claims. While several embodiments have been shown and described, various modifications may be made without departing from the spirit and scope of the present invention.

1. A hybrid polypeptide immunogen comprising a modified ORF0657n sequence segment at least about 100 amino acids in length, wherein said modified sequence segment comprises one or more alterations that increases sequence similarity to SEQ ID NO: 1.

2. The hybrid polypeptide of claim 1, wherein said modified sequence segment comprises at least about 100 amino acids of a modified amino acid sequence selected from the group consisting of SEQ ID NO: 2, SEQ ID NO: 3, SEQ ID NO: 4, SEQ ID NO: 5, and SEQ ID NO: 6, provided that said modified amino acid sequence contains at least 8 amino acid alterations that increase sequence similarity to SEQ ID NO: 1.

3. The hybrid polypeptide of claim 3, wherein said modified amino acid sequence is SEQ ID NO: 2 containing 8 to 100 amino acid alterations that increase sequence similarity to SEQ ID NO: 1.

4. The hybrid polypeptide of claim 2, wherein said modified amino acid sequence has the following sequence:
X¹-AIKNPAI-X²-DK-X³-H-X⁴-APN-X⁵-RPIDFEMX^{-X}⁶-X⁷-
X⁸-G-X⁹-QQFYHYAS-X¹⁰-V-X¹¹-PARVIFT-X¹²-X¹³-K-X¹⁴-
IELGLQ-X¹⁵-X¹⁶-X¹⁷-X¹⁸-W-X¹⁹-KFEVYEGDKKLP-X²⁰-
KLVSYD-X²¹-X²²-KDYAYIRFSVSNGT-X²³-X²⁴-VKIVSSTH-
X²⁵-X²⁶-X²⁷-N-X²⁸-X²⁹-EKYDYTLM-X³⁰-FAQPIYN-X³¹-
X³²-DK-X³³-X³⁴-X³⁵-EEDY-X³⁶-X³⁷-X³⁸-
KLLAPYKKAKTLERQVYEL-X³⁹-K-X⁴⁰-Q-X⁴¹-
KLPEKLKAEYKKKL-X⁴²-X⁴³-T-X⁴⁴-KAL-X⁴⁵-X⁴⁶-QVKSA-
X⁴⁷-TEFQNV-X⁴⁸-PTN-X⁴⁹-K-X⁵⁰-TDLQ-X⁵¹-X⁵²-X⁵³-
X⁵⁴-VV-X⁵⁵-ESVEN-X⁵⁶-ES-X⁵⁷-MDTFV-X⁵⁸-HPIKT-X⁵⁹-
X⁶⁰-LNGKKY-X⁶¹-VM-X⁶²-TTND-X⁶³-YWKDF-X⁶⁴-VEG-
X⁶⁵-RVRT-X⁶⁶-SKD-X⁶⁷-KNN-X⁶⁸-RT-X⁶⁹-IFPY-X⁷⁰-
EGK-X⁷¹-X⁷²-YDAIVKV-X⁷³-VKTI-X⁷⁴-Y-X⁷⁵-GQYHVRI-
X⁷⁶-DK-X⁷⁷-X⁷⁸-X⁷⁹
wherein
X¹ is either E or a D alteration;
X² is either K or an I alteration;
X³ is either D or an E alteration;
X⁴ is either S or a T alteration;
X⁵ is either S or a W alteration;
X⁶-X⁷-X⁸ is either KKD or NDK alterations;
X⁹ is either T or an E alteration;
X¹⁰ is either S or a T alteration;
X¹¹ is either K or an E alteration;
X¹² is either D or a K alteration;
X¹³ is either S or a T alteration;
X¹⁴ is either E or an I alteration;
X¹⁵ is either S or a T alteration;
X¹⁶ is either G or an A alteration;
X¹⁷-X¹⁸ is either KF or ST alterations;
X¹⁹ is either R or a K alteration;
X²⁰ is either I or a V alteration;
X²¹ is either T or an S alteration;
X²² is either V or a D alteration;
X²³ is either K or an R alteration;
X²⁴ is either A or an E alteration;
X²⁵ is either F or a Y alteration;
X²⁶-X²⁷ is either N or GE alterations;
X²⁸-X²⁹ is either KE or IH alterations;
X³⁰ is either E or a V alteration;
X³¹-X³² is either SA or NP alterations;
X³³ is either F or an Y alteration;
X³⁴-X³⁵ is either KT or VD alterations;
X³⁶-X³⁷-X³⁸ is either KAE or NLQ alterations;
X³⁹ is either N or an E alteration;
X⁴⁰ is either I or a L alteration;
X⁴¹ is either D or an E alteration;
X⁴² is either E or a D alteration;
X⁴³ is either D or a Q alteration;
X⁴⁴ is either K or an R alteration;
X⁴⁵ is either D or an A alteration;
X⁴⁶ is either E or a D alteration;
X⁴⁷ is either I or a V alteration;
X⁴⁸ is either Q or a T alteration;
X⁴⁹ is either E or a D alteration;
X⁵⁰ is either M or an L alteration;
X⁵¹ is either D or an E alteration;
X⁵²-X⁵³ is either TK or AH alterations;
X⁵⁴ is either Y or an F alteration;
X⁵⁵ is either Y or an F alteration;
X⁵⁶ is either N or a S alteration;
X⁵⁷ is either M or a V alteration;
X⁵⁸ is either K or an E alteration;
X⁵⁹ is either G or an A alteration;
X⁶⁰ is either M or a T alteration;
X⁶¹ is either M or a V alteration;
X⁶² is either E or a K alteration;
X⁶³ is either D or a S alteration;
X⁶⁴ is either M or an I alteration;
X⁶⁵ is either Q or a K alteration;
X⁶⁶ is either I or a V alteration;
X⁶⁷ is either A or a P alteration;
X⁶⁸ is either T or an S alteration;
X⁶⁹ is either I or a L alteration;
X⁷⁰ is either V or an I alteration;
X⁷¹ is either T or an A alteration;
X⁷² is either L or a V alteration;
X⁷³ is either H or a V alteration;
X⁷⁴ is either D or a G alteration;
X⁷⁵ is either D or an E alteration;
X⁷⁶ is either V or an I alteration;
X⁷⁷ is either E or a D alteration;
X⁷⁸ is either A or an I alteration;
X⁷⁹ is either F or a N alteration;
provided that at least 20 of said alterations are present.

5. The hybrid polypeptide of claim 4, wherein said modified sequence segment comprises at least 200 amino acids of said modified amino acid sequence.

6. The hybrid polypeptide of claim 4, wherein said modified sequence segment comprises said modified amino acid sequence and at least 55 of said alterations are present.

7. The hybrid polypeptide of claim 1, wherein said hybrid polypeptide consists of a sequence selected from the group consisting of SEQ ID NOs: 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, and 43.

8. A method of making a hybrid polypeptide comprising the step of introducing one or more alterations into a ORF0657n sequence segment at least about 100 amino acids in length, wherein at least one of said alterations increases sequence similarity to SEQ ID NO: 1.

9. An immunogen comprising the modified ORF0657n sequence of claim 1 and one or more additional regions or moieties covalently joined to said sequence at the carboxyl terminus or amino terminus, wherein each region or moiety is independently selected from a region or moiety having at least one of the following properties: enhances the immune response, facilitates purification, or facilitates polypeptide stability.

10. A composition able to induce a protective immune response in a patient comprising an immunologically effective amount of the immunogen of claim 1 and a pharmaceutically acceptable carrier.

11. The composition of claim 10, wherein said composition further comprises an adjuvant.

12. A method of inducing a protective immune response in a patient comprising the step of administering to said patient an immunologically effective amount of the immunogen of claim 1.

13. The method of claim 12, wherein said patient is a human.

14. The method of claim 13, wherein said patient is being treated prophylactically against S. aureus infection.

15. A nucleic acid comprising a nucleotide sequence encoding the polypeptide of claim 1.

16. The nucleic acid of claim 15, wherein said nucleic acid is an expression vector and said nucleotide sequence is part of a recombinant gene.

17. A cell comprising the recombinant gene of claim 16, wherein said recombinant gene expresses said nucleic acid sequence in said cell to produce said polypeptide.

18. A method for evaluating the efficacy of an immunogen to produce a protective immune response against Staphylococcus comprising the steps of:
(a) inoculating an animal model with said immunogen to produce an immunized animal model;
(b) challenging said immunized animal model with a Staphylococcus challenge at a potency that provides about 80 to 90% death in said animal model over a period of about 7 to 10 days starting on the first or second day, wherein said Staphylococcus challenge is produced from Staphylococcus grown to stationary phase, and said Staphylococcus challenge is intravenously introduced into said immunized animal model; and
(c) measuring the ability of said immunogen to provide protective immunity.

19. The method of claim 18, wherein said Staphylococcus is Staphylococcus aureus.

20. The method of claim 19, wherein said animal model is a rat or mouse.

21-23. (canceled)


SEQUENCE LISTING (56 sequences)

SEQ ID NO: 1 - protein, 895 aa, Staphylococcus aureus
MNKHHPKLRSFYSIRKSTLGVASVIVSTLFLITSQHQAQAAENTNTSDKISENQNNNATTTQPPKDTNQTQPATQPANTAKNYPAADESLKDAIKDPALENKEHDIGPREQVNFQLLDKNNETQYYHFFSIKDPADVYYTKKKAEVELDINTASTWKKFEVYENNQKLPVRLVSYSPVPEDHAYIRFPVSDGTQELKIVSSTQIDDGEETNYDYTKLVFAKPIYNDPSLVKSDTNDAVVTNDQSSSVASNQTNTNTSNQNTSTINNANNQPQATTNMSQPAQPKSSTNADQASSQPAHETNSNGNTNDKTNESSNQSDVNQQYPPADESLQDAIKNPAIIDKEHTADNWRPIDFQMKNDKGERQFYHYASTVEPATVIFTKTGPIIELGLKTASTWKKFEVYEGDKKLPVELVSYDSDKDYAYIRFPVSNGTREVKIVSSIEYGENIHEDYDYTLMVFAQPITNNPDDYVDEETYNLQKLLAPYHKAKTLERQVYELEKLQEKLPEKYKAEYKKKLDQTRVELADQVKSAVTEFENVTPTNDQLTDLQEAHFVVFESEENSESVMDGFVEHPFYTATLNGQKYVVMKTKDDSYWKDLIVEGKRVTTVSKDPKNNSRTLIFPYIPDKAVYNAIVKVVVANIGYEGQYHVRIINQDINTKDDDTSQNNTSEPLNVQTGQEGKVADTDVAENSSTATNPKDASDKADVIEPESDVVKDADNNIDKDVQHDVDHLSDMSDNNHFDKYDLKEMDTQIAKDTDRNVDKDADNSVGMSSNVDTDKDSNKNKDKVIQLNHIADKNNHTGKAAKLDVVKQNYNNTDKVTDKKTTEHLPSDIHKTVDKTVKTKEKAGTPSKENKLSQSKMLPKTGETTSSQSWWGLYALLGMLALFIPKFRKESK

SEQ ID NO: 2 - protein, 645 aa, Staphylococcus aureus
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 3 - protein, 649 aa, Staphylococcus aureus
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGKVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKNLEKT

SEQ ID NO: 4 - protein, 647 aa, Staphylococcus aureus
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKAVKPAAKATNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSTTPTKVVSTTQNVAKPTASSKTTKDVVQTSAGSSEAKDSAPLQKANIKNTNDGHTQSQNNKNTQENKAKSLPQTGEESNKDMTLPLMALLALSSIVAFVLPRKRKNLEKP

SEQ ID NO: 5 - protein, 645 aa, Staphylococcus aureus
MNKQQKEFKSFYSIRKSSLGVASVAISTLLLLMSNGEAQAAEETGGTNTEAQPKTEAVASPTTTSEKAPETKPVANAVSVSNKEVEAPTSETKEAKEVKEVKAPKETKAVKPATKADNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKREATPATPSKPTPSPVEKESQKQDSQKDDNKQLPSVEKENDASSESGKDKTPATKPAKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKNL

SEQ ID NO: 6 - protein, 645 aa, Staphylococcus aureus
MNKQQKEFKSFYSIRKSSLGVASVAISTLLLLMSNGEAQAAEETGGTNTEAQPKTEAVASPTTTSEKAPETKPVANAVSVSNKEVEAPTSETKEAKEVKEVKAPKETKAVKPATKADNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPAKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKNL

SEQ ID NO: 7 - protein, 654 aa, Staphylococcus aureus
MNKQQKEFKSFYSIRKSSLGVASVAISTLLLLMSNGEAKAAEETGGTITETQPKTEAVASPTTTTEKAPEAKPVANAVSVSNKEVVAPTTETKEAKEVKEVKAPNETKEVKPAAKSDNNTYPILNEELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEETKKALDEQVKSAITEFQNVQPTNEKMTDLQDTKYVVYESVENNESMMDTFVKHPIKTGMLNGKKYMVMETTNDDYWKDFMVEGERVRTISKDAKNNTRTIIFPYVEGKTLYDAIVKVHVKTIDYDGQYHVRIVDKEAFTKANADKSNKKEQQDNSAKKEATPATPSKPTTAPVEKESQKQDSQKDDNKQSPSVEKEIDASSESGKDKTPATKPAKGEVESSSTTPTKVVSATQNVAKPTSASSETTKGVVQTSAGSSEAKDNAPLQKANIKNTNDGHTQSQNNKNTQENKAKSLPQTGEESNKDMTLPLMALLALSSIIAFVLPRKRKNLEKPVRANSS

SEQ ID NO: 8 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIIDKDHSAPNWRPIDFEMKNDKGTQQFYHYASSVEPARVIFTKSKPIIELGLQSGSTWRKFEVYEGDKKLPIKLVSYDTDKDYAYIRFSVSNGTKEVKIVSSTHFGENIHKYDYTLMVFAQPIYNNPDKFVDEEDYNLQKLLAPYKKAKTLERQVYELEKIQDKLPEKLKAEYKKKLEQTKKALAEQVKSAITEFQNVQPTNEKMTDLQDAHYVVYESVENSESMMDTFVEHPIKTGTLNGKKYMVMKTTNDSYWKDFMVEGKRVRTISKDPKNNTRTIIFPYVEGKALYDAIVKVVVKTIDYDGQYHVRIVDKEINTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 9 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKEHSAPNSRPIDFEMKKKDGTQQFYHYASSVKPARVIFTDSKPEIELGLQSGQFWRKFEVYEGDKKLPVKLVSYDTVKDYAYIRFSVSNGTKAVKIVSSTHFNNKEEKYDYTLMEFAQPIYNSADKFKTEEDYKAEKLLAPYKKAKTLERQVYELNKLQEKLPEKLKAEYKKKLEDTKKALDEQVKSAVTEFQNVQPTNDKMTDLQDTKYVVYESVENNESMMDTFVKHPIKTGMLNGKKYMVMETTNDDYWKDFMVEGQRVRTISKDAKNNTRTLIFPYIEGKTLYDAIVKVHVKTIDYEGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 10 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELRDAIKNPAIKDKEHTAPNSRPIDFEMKKKDGTQQFYHYASTVKPARVIFTDTKPEIELGLQTAQFWKKFEVYEGDKKLPVKLVSYDSVKDYAYIRFSVSNGTRAVKIVSSTHYNNKEEKYDYTLMEFAQPIYNSADKYKTEEDYKAEKLLAPYKKAKTLERQVYELNKLQDKLPEKLKAEYKKKLDDTRKALDDQVKSAVTEFQNVQPTNDKLTDLQETKFVVFESVENNESVMDTFVKHPIKTAMLNGKKYVVMETTNDDYWKDFIVEGQRVRTVSKDAKNNSRTLIFPYIEGKTVYDAIVKVHVKTIDYEGQYHVRIIDKDAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 11 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKEVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 12 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNWRPIDFEMKKKDGTQQFYHYASSVEPARVIFTDSKPEIELGLQSGQFWRKFEVYEGDKKLPIKLVSYDTVKDYAYIRFSVSNGTKEVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 13 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNWRPIDFEMKKKDGTQQFYHYASSVEPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELEKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 14 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNWRPIDFEMKKKDGTQQFYHYASSVEPARVIFTDSKPEIELGLQSGQFWRKFEVYEGDKKLPIKLVSYDTVKDYAYIRFSVSNGTKEVKIVSSTHFNNKEEKYDYTLMVFAQPIYNSADKFKTEEDYKAEKLLAPYKKAKTLERQVYELNKIQDKLPEKLKAEYKKKLEDTKKALAEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 15 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNWRPIDFEMKKKDGTQQFYHYASSVEPARVIFTDSKPEIELGLQSGQFWRKFEVYEGDKKLPIKLVSYDTVKDYAYIRFSVSNGTKEVKIVSSTHFNNKEEKYDYTLMEFAQPIYNSADKFKTEEDYKAEKLLAPYKKAKTLERQVYELEKIQDKLPEKLKAEYKKKLEDTKKALAEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 16 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNWRPIDFEMKKKDGTQQFYHYASSVEPARVIFTKSKPEIELGLQSGQFWRKFEVYEGDKKLPIKLVSYDTDKDYAYIRFSVSNGTKEVKIVSSTHFNNKEEKYDYTLMEFAQPIYNSADKFKTEEDYKAEKLLAPYKKAKTLERQVYELEKIQDKLPEKLKAEYKKKLEDTKKALAEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 17 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNWRPIDFEMKKKDGTQQFYHYASSVEPARVIFTKSKPEIELGLQSGSTWRKFEVYEGDKKLPIKLVSYDTDKDYAYIRFSVSNGTKEVKIVSSTHFNNKEEKYDYTLMVFAQPIYNSADKFKTEEDYKAEKLLAPYKKAKTLERQVYELEKIQDKLPEKLKAEYKKKLEDTKKALAEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 18 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNWRPIDFEMKNDKGTQQFYHYASSVEPARVIFTKSKPIIELGLQSGQFWRKFEVYEGDKKLPIKLVSYDTDKDYAYIRFSVSNGTKEVKIVSSTHFNNKEEKYDYTLMVFAQPIYNSADKFKTEEDYKAEKLLAPYKKAKTLERQVYELEKIQDKLPEKLKAEYKKKLEDTKKALAEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 19 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNWRPIDFEMKNDKGTQQFYHYASSVEPARVIFTKSKPIIELGLQSGQFWRKFEVYEGDKKLPIKLVSYDTDKDYAYIRFSVSNGTKEVKIVSSTHFNNKEEKYDYTLMVFAQPIYNSADKFKTEEDYKAEKLLAPYKKAKTLERQVYELEKIQDKLPEKLKAEYKKKLEQTKKALAEQVKSAITEFQNVQPTNEKMTDLQDAHYVVYESVENSESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 20 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNWRPIDFEMKNDKGTQQFYHYASSVEPARVIFTKSKPIIELGLQSGQFWRKFEVYEGDKKLPIKLVSYDTDKDYAYIRFSVSNGTKEVKIVSSTHFNNKEEKYDYTLMVFAQPIYNSADKFKTEEDYKAEKLLAPYKKAKTLERQVYELEKIQDKLPEKLKAEYKKKLEQTKKALAEQVKSAITEFQNVQPTNEKMTDLQDAHYVVYESVENSESMMDTFVKHPIKTGMLNGKKYMVMKTTNDDYWKDFMVEGKRVRTISKDAKNNTRTIIFPYVEGKALYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 21 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNWRPIDFEMKNDKGTQQFYHYASSVEPARVIFTKSKPIIELGLQSGQFWRKFEVYEGDKKLPIKLVSYDTDKDYAYIRFSVSNGTKEVKIVSSTHFNNKEEKYDYTLMVFAQPIYNSADKFKTEEDYKAEKLLAPYKKAKTLERQVYELEKIQDKLPEKLKAEYKKKLEQTKKALAEQVKSAITEFQNVQPTNEKMTDLQDAHYVVYESVENSESMMDTFVKHPIKTGTLNGKKYMVMKTTNDDYWKDFMVEGKRVRTISKDAKNNTRTIIFPYVEGKALYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 22 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNWRPIDFEMKNDKGTQQFYHYASSVEPARVIFTKSKPIIELGLQSGQFWRKFEVYEGDKKLPIKLVSYDTDKDYAYIRFSVSNGTKEVKIVSSTHFNNKEEKYDYTLMVFAQPIYNSADKFKTEEDYKAEKLLAPYKKAKTLERQVYELEKIQDKLPEKLKAEYKKKLEQTKKALAEQVKSAITEFQNVQPTNEKMTDLQDAHYVVYESVENSESMMDTFVEHPIKTGTLNGKKYMVMKTTNDDYWKDFMVEGKRVRTISKDAKNNTRTIIFPYVEGKALYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 23 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIIDKDHSAPNWRPIDFEMKNDKGTQQFYHYASSVEPARVIFTKSKPIIELGLQSGQFWRKFEVYEGDKKLPIKLVSYDTDKDYAYIRFSVSNGTKEVKIVSSTHFNNKEEKYDYTLMVFAQPIYNSADKFKTEEDYKAEKLLAPYKKAKTLERQVYELEKIQDKLPEKLKAEYKKKLEQTKKALAEQVKSAITEFQNVQPTNEKMTDLQDAHYVVYESVENSESMMDTFVEHPIKTGTLNGKKYMVMKTTNDDYWKDFMVEGKRVRTISKDAKNNTRTIIFPYVEGKALYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 24 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIIDKDHSAPNWRPIDFEMKNDKGTQQFYHYASSVEPARVIFTKSKPIIELGLQSGQFWRKFEVYEGDKKLPIKLVSYDTDKDYAYIRFSVSNGTKEVKIVSSTHFNNKEEKYDYTLMVFAQPIYNSADKFKTEEDYNAEKLLAPYKKAKTLERQVYELEKIQDKLPEKLKAEYKKKLEQTKKALAEQVKSAITEFQNVQPTNEKMTDLQDAHYVVYESVENSESMMDTFVEHPIKTGTLNGKKYMVMKTTNDDYWKDFMVEGKRVRTISKDAKNNTRTIIFPYVEGKALYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 25 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIIDKDHSAPNWRPIDFEMKNDKGTQQFYHYASSVEPARVIFTKSKPIIELGLQSGSTWRKFEVYEGDKKLPIKLVSYDTDKDYAYIRFSVSNGTKEVKIVSSTHFNNKEEKYDYTLMVFAQPIYNSADKFKTEEDYNLQKLLAPYKKAKTLERQVYELEKIQDKLPEKLKAEYKKKLEQTKKALAEQVKSAITEFQNVQPTNEKMTDLQDAHYVVYESVENSESMMDTFVEHPIKTGTLNGKKYMVMKTTNDDYWKDFMVEGKRVRTISKDAKNNTRTIIFPYVEGKALYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 26 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIIDKDHSAPNWRPIDFEMKNDKGTQQFYHYASSVEPARVIFTKSKPIIELGLQSGSTWRKFEVYEGDKKLPIKLVSYDTDKDYAYIRFSVSNGTKEVKIVSSTHFNNKEEKYDYTLMVFAQPIYNSADKFKTEEDYNLQKLLAPYKKAKTLERQVYELEKIQDKLPEKLKAEYKKKLEQTKKALAEQVKSAITEFQNVQPTNEKMTDLQDAHYVVYESVENSESMMDTFVEHPIKTGTLNGKKYMVMKTTNDDYWKDFMVEGKRVRTISKDAKNNTRTIIFPYVEGKALYDAIVKVVVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 27 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIIDKDHSAPNWRPIDFEMKNDKGTQQFYHYASSVEPARVIFTKSKPIIELGLQSGSTWRKFEVYEGDKKLPIKLVSYDTDKDYAYIRFSVSNGTKEVKIVSSTHFNNKEEKYDYTLMVFAQPIYNSADKFKTEEDYNLQKLLAPYKKAKTLERQVYELEKIQDKLPEKLKAEYKKKLEQTKKALAEQVKSAITEFQNVQPTNEKMTDLQDAHYVVYESVENSESMMDTFVEHPIKTGTLNGKKYMVMKTTNDDYWKDFMVEGKRVRTISKDAKNNTRTIIFPYVEGKALYDAIVKVVVKTIDYDGQYHVRIVDKEINTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 28 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIIDKDHSAPNWRPIDFEMKNDKGTQQFYHYASSVEPARVIFTKSKPIIELGLQSGSTWRKFEVYEGDKKLPIKLVSYDTDKDYAYIRFSVSNGTKEVKIVSSTHFNNKEEKYDYTLMVFAQPIYNSADKFKTEEDYNLQKLLAPYKKAKTLERQVYELEKIQDKLPEKLKAEYKKKLEQTKKALAEQVKSAITEFQNVQPTNEKMTDLQDAHYVVYESVENSESMMDTFVEHPIKTGTLNGKKYMVMKTTNDDYWKDFMVEGKRVRTISKDPKNNTRTIIFPYVEGKALYDAIVKVVVKTIDYDGQYHVRIVDKEINTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 29 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIIDKDHSAPNWRPIDFEMKNDKGTQQFYHYASSVEPARVIFTKSKPIIELGLQSGSTWRKFEVYEGDKKLPIKLVSYDTDKDYAYIRFSVSNGTKEVKIVSSTHFGNKEEKYDYTLMVFAQPIYNNPDKFKTEEDYNLQKLLAPYKKAKTLERQVYELEKIQDKLPEKLKAEYKKKLEQTKKALAEQVKSAITEFQNVQPTNEKMTDLQDAHYVVYESVENSESMMDTFVEHPIKTGTLNGKKYMVMKTTNDDYWKDFMVEGKRVRTISKDPKNNTRTIIFPYVEGKALYDAIVKVVVKTIDYDGQYHVRIVDKEINTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 30 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAVTEFQNVQPTNDKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 31 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAVTEFQNVQPTNDKMTDLQDTKYVVYESVENNESMMDTFVKHPIKTGMLNGKKYMVMETTNDDYWKDFMVEGQRVRTISKDAKNNTRTIIFPYIEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 32 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKEHSAPNSRPIDFEMKKKDGTQQFYHYASSVKPARVIFTDSKPEIELGLQSGQFWRKFEVYEGDKKLPVKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAVTEFQNVQPTNDKMTDLQDTKYVVYESVENNESMMDTFVKHPIKTGMLNGKKYMVMETTNDDYWKDFMVEGQRVRTISKDAKNNTRTIIFPYIEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 33 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKEHSAPNSRPIDFEMKKKDGTQQFYHYASSVKPARVIFTDSKPEIELGLQSGQFWRKFEVYEGDKKLPVKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAVTEFQNVQPTNDKMTDLQDTKYVVYESVENNESMMDTFVKHPIKTGMLNGKKYMVMETTNDDYWKDFMVEGQRVRTISKDAKNNTRTLIFPYIEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 34 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKEHSAPNSRPIDFEMKKKDGTQQFYHYASSVKPARVIFTDSKPEIELGLQSGQFWRKFEVYEGDKKLPVKLVSYDTVKDYAYIRFSVSNGTKAVKIVSSTHFNNKEEKYDYTLMEFAQPIYNSADKFKTEEDYKAEKLLAPYKKAKTLERQVYELNKLQEKLPEKLKAEYKKKLEDTKKALDEQVKSAVTEFQNVQPTNDKMTDLQDTKYVVYESVENNESMMDTFVKHPIKTGMLNGKKYMVMETTNDDYWKDFMVEGQRVRTISKDAKNNTRTLIFPYIEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 35 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPVKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 36 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPVKLVSYDSVKDYAYIRFSVSNGTRAVKIVSSTHYNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 37 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDTKPEIELGLQSGQFWRKFEVYEGDKKLPVKLVSYDSVKDYAYIRFSVSNGTRAVKIVSSTHYNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 38 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASTVKPARVIFTDTKPEIELGLQSGQFWRKFEVYEGDKKLPVKLVSYDSVKDYAYIRFSVSNGTRAVKIVSSTHYNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 39 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELRDAIKNPAIKDKEHTAPNSRPIDFEMKKKDGTQQFYHYASTVKPARVIFTDTKPEIELGLQSGQFWRKFEVYEGDKKLPVKLVSYDSVKDYAYIRFSVSNGTRAVKIVSSTHYNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 40 - protein, 645 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELRDAIKNPAIKDKEHTAPNSRPIDFEMKKKDGTQQFYHYASTVKPARVIFTDTKPEIELGLQTAQFWRKFEVYEGDKKLPVKLVSYDSVKDYAYIRFSVSNGTRAVKIVSSTHYNNKEEKYDYTLMEFAQPIYNSADKYKTEEDYKAEKLLAPYKKAKTLERQVYELNKLQDKLPEKLKAEYKKKLDDTKKALDDQVKSAVTEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 41 - protein, 639 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIIDKDHSAPNSRPIDFEMKKKDGTQQFYHYASSVKPARVIFTKTGPVIELGLKTASTWRKFEVYEGDKKLPIKLVSYDTVKDYAYIRFPVSNGTRDVKIVSSIEYGENIHEDYDYTLMVFAQPITNNPDDYVDEETYNLQKLLAPYHKAKTLERQVYELNKIQDKLPEKLKAEYKKKLEDTKKALDEQVKSAITEFQNVQPTNEKMTDLQDTKYVVFESEENSESVMDGFVEHPFYTATLNGQKYVVMKTKDDSYWKDLIVEGKRVTTVSKDPKNNSRTIIFPYVEGKAVYNAIVKVVVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFV

SEQ ID NO: 42 - protein, 890 aa, Artificial Sequence
  Hybrid sequence
MNKHHPKLRSFYSIRKSTLGVASVIVSTLFLITSQHQAQAAENTNTSDKISENQNNNATTTQQPKDTNQTQPATQPVITAKNYPAADESLKDAIKDPALENKEHDIGPREQVNFQLLDKNNETQYYHFFSIKDPADVYYTKKKAEVELDINTASTWKKFEVYENNQKLPVRLVSYSPVPEDHAYIRFPVSDGTQELKIVSSTQIDDGEETNYDYTKLVFAKPIYNDPSLVKSDTNDAVVTNDQSSSDASNQTNTNTSNQNTSTTNNANNQPQATTNMSQPAQPKSSANADQASSQPAHETNSNGNTNDKTNESSNQSDVNQQYPPADESLQDAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIINQDINTKDDDTSQNNTSEPLNVQTGQEGKVADTDVAENSSTATNPKDASDKADVIEPESDVVKDADNNIDKDVQHDVDHLSDMSDNNHFDKYDLKEMDTQIAKDTDRNVDNSVGMSSNVDTDKDSNKNKDKVIQLAHIADKNNHTGKAAKLDVVKQNYNNTDKVTDKKTTEHLPSDIHKTVDKTVKTKEKAGTPSKENKLSQSKMLPKTGETTSSQSWWGLYALLGMLALFIPKFRKESK

SEQ ID NO: 43 - protein, 639 aa, Artificial Sequence
  Hybrid sequence
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIIDKEHTADNWRPIDFQMKNDKGERQFYHYASTVEPATVIFTKTGPVIELGLKTASTWKKFEVYEGDKKLPVELVSYDSDKDYAYIRFPVSNGTRDVKIVSSIEYGENIHEDYDYTLMVFAQPITNNPDDYVDEETYNLQKLLAPYHKAKTLERQVYELEKLQEKLPEKYKAEYKKKLDQTRVELADQVKSAVTEFENVTPTNDQLTDLQEAHFVVFESEENSESVMDGFVEHPFYTATLNGQKYVVMKTKDDSYWKDLIVEGKRVTTVSKDPKNNSRTLIFPYIPDKAVYNAIVKVVVANIGYEGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFV

SEQ ID NO: 44 - protein, 654 aa, Artificial Sequence
  0657 mutated protein
MGNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKNLEHHHHHH

SEQ ID NO: 45 - DNA, 1938 bp, Artificial Sequence
  0657 cDNA
atgaacaaacagcaaaaagaatttaaatcattttattcaattagaaagtcatcactaggcgttgcatctgtagcaattagtacacttttattattaatgtcaaatggcgaagcacaagcagcagctgaagaaacaggtggtacaaatacagaagcacaaccaaaaactgaagcagttgcaagtccaacaacaacatctgaaaaagctccagaaactaaaccagtagctaatgctgtctcagtatctaataaagaagttgaggcccctacttctgaaacaaaagaagctaaagaagttaaagaagttaaagcccctaaggaaacaaaagaagttaaaccagcagcaaaagccactaacaatacatatcctattttgaatcaggaacttagagaagcgattaaaaaccctgcaataaaagacaaagatcatagcgcaccaaactctcgtccaattgattttgaaatgaaaaagaaagatggaactcaacagttttatcattatgcaagttctgttaaacctgctagagttattttcactgattcaaaaccagaaattgaattaggattacaatcaggtcaattttggagaaaatttgaagtttatgaaggtgacaaaaagttgccaattaaattagtatcatacgatactgttaaagattatgcttacattcgcttctctgtatcaaacggaacaaaagctgttaaaattgttagttcaacacacttcaataacaaagaagaaaaatacgattacacattaatggaattcgcacaaccaatttataacagtgcagataaattcaaaactgaagaagattataaagctgaaaaattattagcgccatataaaaaagcgaaaacactagaaagacaagtttatgaattaaataaaattcaagataaacttcctgaaaaattaaaggctgagtacaagaagaaattagaggatacaaagaaagctttagatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccaactaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattattagctttaagtagcatcgttgcattcgtattacctagaaaacgtaaaaactaa

SEQ ID NO: 46 - DNA, 39 bp, Artificial Sequence
  ORF0657nF primer
aaccggttttccatggggaacaaacagcaaaaagaattt

SEQ ID NO: 47 - protein, 12 aa, Artificial Sequence
  ORF0657nF encoded amino acid
NRFSMGNKQQKE

SEQ ID NO: 48 - DNA, 38 bp, Artificial Sequence
  ORF0657nR primer sequence
gtattacctagaaaacgtaaaaacctcgagaaaccggt

SEQ ID NO: 49 - protein, 12 aa, Artificial Sequence
  ORF0657nR encoded amino acid
VLPRKRKNLEKP

SEQ ID NO: 50 - DNA, 16 bp, Artificial Sequence
  Primer
ctggccgtcgttttac

SEQ ID NO: 51 - DNA, 17 bp, Artificial Sequence
  Primer
caggaaacagctatgac

SEQ ID NO: 52 - DNA, 1938 bp, Artificial Sequence
  cDNA encoding ORF0657n protein of SEQ 3
atgaacaaacagcaaaaagaatttaaatcattttattcaattagaaagtcatcactaggcgttgcatctgtagcaattagtacacttttattattaatgtcaaatggcgaagcacaagcagcagctgaagaaacaggtggtacaaatacagaagcacaaccaaaaactgaagcagttgcaagtccaacaacaacatctgaaaaagctccagaaactaaaccagtagctaatgctgtctcagtatctaataaagaagttgaggctcctacttctgaaacaaaagaagctaaagaagttaaagaagttaaagcccctaaggaaacaaaagaagttaaaccagcagcaaaagccactaacaatacatatcctattttgaatcaggaacttagagaagcgattaaaaaccctgcaataaaagacaaagatcatagcgcaccaaactctcgtccaattgattttgaaatgaaaaagaaagatggaactcaacagttttatcattatgcaagttctgttaaacctgctagagttattttcactgattcaaaaccagaaattgaattaggattacaatcaggtcaattttggagaaaatttgaagtttatgaaggtgacaaaaagttgccaattaaattagtatcatacgatactgttaaagattatgcttacattcgcttctctgtatcaaacggaacaaaagctgttaaaattgttagttcaacacacttcaataacaaagaagaaaaatacgattacacattaatggaattcgcacaaccaatttataacagtgcagataaattcaaaactgaagaagattataaagctgaaaaattattagcgccatataaaaaagcgaaaacactagaaagacaagtttatgaattaaataaaattcaagataaacttcctgaaaaattaaaggctgagtacaagaagaaattagaggatacaaagaaagctttagatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccaactaaaggtaaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattattagctttaagtagcatcgttgcattcgtattacctagaaaacgtaaaaactaa

SEQ ID NO: 53 - DNA, 1938 bp, Artificial Sequence
  cDNA encoding ORF0657n protein of SEQ 4
atgaacaaacagcaaaaagaatttaaatcattttattcaattagaaagtcatcactaggcgttgcatctgtagcgattagtacacttttattattaatgtcaaatggcgaagcacaagcagcagctgaagaaacaggtggtacaaatacagaagcacaaccaaaaactgaagcagttgcaagtccaacaacaacatctgaaaaagctccagaaactaaaccagtagctaatgctgtctcagtatctaataaagaagttgaggcccctacttctgaaacaaaagaagctaaagaagttaaagaagttaaagcccctaaggaaacaaaagcagttaaaccagcagcaaaagccactaacaatacatatcctattttgaatcaggaacttagagaagcgattaaaaaccctgcaataaaagataaagatcatagcgcaccaaactctcgtccaattgattttgaaatgaaaaaagaaaatggtgagcaacaattttatcattatgccagctctgttaaacctgctagagttattttcactgattcaaaaccagaaattgaattaggattacaatcaggtcaattttggagaaaatttgaagtttatgaaggtgacaaaaagttgccaattaaattagtatcatacgatactgttaaagattacgcttacattcgcttctctgtttcaaatggaacaaaagccgttaaaattgtaagttcaactcacttcaataacaaagaagaaaaatacgattacacattaatggaattcgcacaaccaatttataacagtgcagataaattcaaaactgaagaagattataaagctgaaaaattattagcgccatataaaaaagcgaaaacactagaaagacaagtttatgaattaaataaaattcaagataaacttcctgaaaaattaaaggctgagtacaagaagaaattagaggatacaaagaaagctttagatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccaactaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattactagctttaagtagcatcgttgcattcgtattacctagaaaacgtaaaaactaa

SEQ ID NO: 54 - DNA, 1935 bp, Artificial Sequence
  cDNA encoding ORF0657n protein of SEQ 5
atgaacaaacagcaaaaagaatttaaatcattttattcaattagaaagtcatcactaggcgttgcatctgtagcgattagtacacttttattattaatgtcaaatggcgaagcacaagcagctgaagaaacaggtggtacaaatacagaagcacaaccaaaaactgaagcagttgcaagtccaacaacaacatctgaaaaagctccagaaactaaaccagtagctaatgctgtctcagtatctaataaagaagttgaggcccctacttctgaaacaaaagaagctaaagaagttaaagaagttaaagcccctaaggaaacaaaagcagttaaaccagcaacaaaagccgataacaatacatatcctattttgaatcaggaacttagagaagcgattaaaaaccctgcaataaaagataaagatcatagcgcaccaaactctcgtccaattgattttgaaatgaaaaaagaaaatggtgagcaacaattttatcattatgccagctctgttaaacctgctagagttattttcactgattcaaaaccagaaattgaattaggattacaatcaggtcaattttggagaaaatttgaagtttatgagggtgacaaaaagttgccaattaaattagtatcatacgatactgttaaagattatgcttacattcgtttctctgtatcaaatggaacaaaagctgttaaaattgttagttcaacacacttcaataacaaagaagaaaaatacgattacacattaatggaattcgcacaaccaatttataacagtgcagataaattcaaaactgaagaagattataaagctgaaaaattattagcgccatataaaaaagcgaaaacactagaaagacaagtttatgaattaaataaaattcaagataaacttcctgaaaaattaaaggctgagtacaagaagaaattagaggatacaaagaaagctttagatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagagggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccagctaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattattagctttaagtagcatcgttgcattcgtattacctagaaaacgtaaaaactaa

SEQ ID NO: 55 - DNA, 1935 bp, Artificial Sequence
  cDNA encoding ORF0657n protein of SEQ 6
atgaacaaacagcaaaaagaatttaaatcattttattcaattagaaagtcatcactaggcgttgcatctgtagcgattagtacacttttattattaatgtcaaatggcgaagcacaagcagctgaagaaacaggtggtacaaatacagaagcacaaccaaaaactgaagcagttgcaagtccaacaacaacatctgaaaaagctccagaaactaaaccagtagctaatgctgtctcagtatctaataaagaagttgaggcccctacttctgaaacaaaagaagctaaagaagttaaagaagttaaagcccctaaggaaacaaaagcagttaaaccagcaacaaaagccgataacaatacatatcctattttgaatcaggaacttagagaagcgattaaaaaccctgcaataaaagataaagatcatagcgcaccaaactctcgtccaattgattttgaaatgaaaaaagaaaatggtgagcaacaattttatcattatgccagctctgttaaacctgctagagttattttcactgattcaaaaccagaaattgaattaggattacaatcaggtcaattttggagaaaatttgaagtttatgagggtgacaaaaagttgccaattaaattagtatcatacgatactgttaaagattatgcttacattcgtttctctgtatcaaatggaacaaaagctgttaaaattgttagttcaacacacttcaataacaaagaagaaaaatacgattacacattaatggaattcgcacaaccaatttataacagtgcagataaattcaaaactgaagaagattataaagctgaaaaattattagcgccatataaaaaagcgaaaacactagaaagacaagtttatgaattaaataaaattcaagataaacttcctgaaaaattaaaggctgagtacaagaagaaattagaggatacaaagaaagctttagatgagcaagtgaaatcagctattactgaattccaaaatgtacaaccaacaaatgaaaaaatgactgatttgcaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccagctaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattattagctttaagtagcatcgttgcattcgtattacctagaaaacgtaaaaactaa

SEQ ID NO: 56 - DNA, 1934 bp, Artificial Sequence
  cDNA encoding ORF0657n protein of SEQ 7
atgaacaaacagcaaaaagaatttaaatcattttattcaattagaaagtcatcactaggcgttgcatctgtagcgattagtacacttttattattaatgtcaaatggcgaagcaaaagcagctgaagaaacaggtggtacaattacagaaacacaaccaaaaactgaagcagttgcaagtccaacaacaacaactgaaaaagctccagaagctaaaccagtagctaatgctgtctccgtatctaataaagaagttgtggcccctactactgaaacaaaagaagctaaagaagttaaagaagttaaagcccctaatgaaacaaaagaagttaaaccagcagcaaaatccgataacaatacatatcctattttgaatgaggaacttagagaagcgattaaaaacccagcaataaaagataaagatcatagcgcaccaaactctcgtccaattgattttgaaatgaaaaagaaagatggcactcaacagttttatcattatgcaagttctgttaaacctgctagagttattttcactgattcaaaaccagaaattgaattaggattacaatcaggtcaattttggagaaaatttgaagtttatgaaggtgacaaaaaattaccaatcaaattagtatcatatgatactgttaaagactatgcttacattcgcttctctgtttcaaatggaacaaaagcagttaaaatcgtaagttcaactcacttcaataataaagaagaaaaatatgattacacgttaatggaattcgcgcaaccgatttataacagtgcagataaattcaaaactgaagaagattacaaagctgaaaaattattagcgccatataaaaaagcgaaaacactagaaagacaagtttatgaattaaataaaattcaagataaacttcccgaaaaattgaaagctgaatacaagaagaaattagaagagactaaaaaagctttagatgaacaagtgaaatccgcaataactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtaatggaaactactaatgacgattactggaaagatttcatggttgaaggtgaacgtgttagaacaattagcaaagatgctaaaaataacactagaacgattattttcccatatgttgaaggtaaaaccctatatgatgctatcgttaaagttcacgtaaaaacgattgattatgatggacaataccatgtcagaatcgttgataaagaagcatttactaaagccaatgccgataaatctaataaaaaggaacaacaagacaactcagctaagaaggaagctacaccagctacgcctagcaaaccaacaacagcacctgttgaaaaagaatcacaaaacaagacagccaaaaagatgacaataaacaatcaccaagtgttgaaaaagaaattgatgcatctagtgagtcaggtaaagacaaaacgcctgctacaaaaccagctaaaggtgaagtagaatcaagtagtacaactccaactaaggtagtatctgcgactcaaaatgttgcaaaacctacatctgcttcttcagaaacaacaaaaggtgttgttcaaacttcagcaggttctagcgaagcaaaagataatgctccattacaaaaagcaaacattaaaaacacaaatgatggacatactcaaagccaaaacaataaaaatacacaagaaaataaagctaaatcattaccacaaactggtgaagaatcgaataaagatatgacattaccattaatggcattattagctttaagcagcatcattgcattcgtattacctagaaaacgtaaaaactaa